(19) United States
(12) Patent Application Publication
(10) Pub. No.: US 2010/0098657 A1
(43) Pub. Date: Apr. 22, 2010

(54) Method of Treating Cancer with Immunomodulatory Compounds and IgG

(76) Inventors: Peter H. Schafer, Somerset, NJ (US); Justin B. Bartlett, Warren, NJ (US)

Correspondence Address: JONES DAY, 222 EAST 41ST ST, NEW YORK, NY, 10017, US

(21) Appl. No.: 12/341,997

(22) Filed: Dec. 22, 2008

Related U.S. Application Data
(60) Provisional application No. 61/009,347, filed on Dec. 27, 2007.

(51) Int. Cl. A61K 39/395; A61K 38/20

(52) U.S. Cl. 424/85.2; 424/130.1; 424/133.1

(57) ABSTRACT

Provided herein relates to the field of cancer and its treatment by administering immunomodulatory compounds in combination with other compounds. In particular, a combination of an immunomodulatory compound and an antibody is provided.

This application claims priority to U.S. Provisional Application No. 61/009,347, filed Dec. 27, 2007, the entirety of which is incorporated herein by reference.

### 1. FIELD

Provided herein are methods for treatment of cancer by administering immunomodulatory compounds in combination with other compounds.

### 2. BACKGROUND

Many types of cancer have been described in detail in the medical literature. Examples includes cancer of the blood, bone, lung, colon, rectum, prostate, breast, brain, and intestine. The incidence of cancer continues to climb as the general population ages and as new cancers develop. A demand exists for new and effective therapies that can be used to treat patients with cancer.

Current cancer therapy may involve such methods as surgery, chemotherapy, hormonal therapy, and radiation therapy. Each of these approaches has drawbacks. Surgery, for example, may be contraindicated due to the health of a patient, and may not be effective in completely removing the cancer. Radiation therapy often elicits serious side effects. Hormonal therapy is rarely effective as a single agent.

Currently used chemotherapy agents typically act by inhibiting DNA synthesis, either directly or indirectly by inhibiting the biosynthesis of deoxyribonucleotide triphosphate precursors to prevent DNA replication and concomitant cell division (Gilman et al., 2001, Goodman and Gilman's: The Pharmacological Basis of Therapeutics, Tenth Ed.; McGraw Hill, New York). Most chemotherapeutic agents are toxic, and chemotherapy causes significant, dangerous side effects including severe nausea, bone marrow depression, and immunosuppression. Additionally, tumor cells can be resistant to or can develop resistance to the chemotherapeutic agents, rendering many cancers to be refractory to standard chemotherapeutic treatment protocols.

In addition to the above-described cancer therapies, several types of antibodies have also been developed to treat cancer patients. Several monoclonal antibodies have been found to be particularly useful in cancer treatment. The anti-CD20 antibody Rituximab, for example, has direct anti-lymphoma activity. These antibodies can participate in the process of targeted cell lysis, which is a mechanism that utilizes antibodies to target certain cells for destruction. Typically, a cell that is to be targeted for destruction is first coated with antibodies that recognize certain cell surface proteins. Cells that are coated with these antibodies can then be targeted by a specialized cell termed a natural killer cell (NK cell). Fc receptors on the surface of the NK cells can recognize bound antibody on the target cells, signaling the NK cell to kill the target cells. During the attack, the NK cells release cytoplasmic granules containing perforin and granzymes. This method of targeting and killing antibody coated cells is termed “antibody dependent cell mediated cytotoxicity” (“ADCC”).

The immunomodulatory compounds, including compounds known as IMiDs® available from Celgene Corporation, are a group of compounds that can be useful to treat several types of human diseases, including certain cancers. These compounds can be prepared synthetically, or can be obtained commercially.

### 3. SUMMARY

Provided herein are methods for treatment of cancer by administering immunomodulatory compounds in combination with other compounds, such as IgG or antibodies.

In some embodiments, a method of treating cancer in a patient is provided. The method comprises administering an immunomodulatory compound, immunoglobulin G (IgG).

In another embodiment, provided herein is a method of treating cancer in a subject comprising administering an immunomodulatory compound prior to administration of antibodies such as, but not limited to, Rituximab, Trastuzumab, and Cetuximab. In some embodiments, the method may also comprise administering cytokines such as IL-2 or IL-12, and/or human serum IgG to the subject.

### 4. BRIEF DESCRIPTION OF THE DRAWINGS

FIG. 1 is a line graph showing the effect of the administration of various concentrations of lenalidomide with or without administration of IgG, IL-2, and IL-12 to NK cells on in vitro IFN-γ production (pg/ml). IFN-γ indicates interferon-gamma; IgG, immunoglobulin G; IL-2, interleukin-2.

FIG. 2 is a series of line graphs comparing the percentage of ADCC-mediated NK cell cytotoxicity after administration of various concentrations of lenalidomide with or without Rituximab, and either IL-2 (left panel) or IL-12 (right panel) in cells of the following NHL cell lines: FIG. 2A: Namalwa cells; FIG. 2B: Raji cells; FIG. 2C: Farage cells.

FIG. 3 is a line graph showing the effector to target ratio vs. the percentage of NK-mediated cytotoxicity in Rituximab-coated CD20⁺ Jeko-1 cells (mantle cell lymphoma). The cells were treated with lenalidomide alone (□), Rituximab alone (⋄), or lenalidomide plus Rituximab (∘).

FIG. 4 is a line graph showing the effector to target ratio vs. the percentage of monocyte mediated lysis of Rituximab-coated Farage cells. The cells were treated with Rituximab alone (∘), Rituximab plus lenalidomide (⋄), or Rituximab, lenalidomide, and anti-IL-12 antibody ().

FIG. 5 is a series of bar graphs demonstrating the enhancement of ADCC by lenalidomide and the increased NK cell expression of various chemokines.

FIG. 5A illustrates the amount of Granulocyte-macrophage colony-stimulating factor (GM-CSF) produced in cells treated with Interleukin-2 (IL-2), immunoglobulin G (IgG), lenalidomide (0.1 μM, 1 μM, or 10 μM), or combinations of each.

FIG. 5B illustrates the amount of IP-10 produced in cells treated with Interleukin-2 (IL-2), immunoglobulin G (IgG), lenalidomide (0.1 μM, 1 μM, or 10 μM), or combinations of each.

FIG. 5C illustrates the amount of MIP-1β produced in cells treated with Interleukin-2 (IL-2), immunoglobulin G (IgG), lenalidomide (0.1 μM, 1 μM, or 10 μM), or combinations of each.

FIG. 5D illustrates the amount of IL-6 produced in cells treated with Interleukin-2 (IL-2), immunoglobulin G (IgG), lenalidomide (0.1 μM, 1 μM, or 10 μM), or combinations of each.

FIG. 5E illustrates the amount of monocyte chemotactic protein-1 (MCP-1) produced in cells treated with Interleukin-2 (IL-2), immunoglobulin G (IgG), lenalidomide (0.1 μM, 1 μM, or 10 μM), or combinations of each.

FIG. 5F illustrates the amount of RANTES produced in cells treated with Interleukin-2 (IL-2), immunoglobulin G (IgG), lenalidomide (0.1 μM, 1 μM, or 10 μM), or combinations of each.

FIG. 5G illustrates the amount of IL-8 produced in cells treated with Interleukin-2 (IL-2), immunoglobulin G (IgG), lenalidomide (0.1 μM, 1 μM, or 10 μM), or combinations of each.

FIG. 5H illustrates the amount of MIP-1α produced in cells treated with Interleukin-2 (IL-2), immunoglobulin G (IgG), lenalidomide (0.1 μM, 1 μM, or 10 μM), or combinations of each.

FIG. 6A is a bar graph comparing the percentage of CD56⁺ NK cells expressing Fas-L in cells treated with DMSO alone (control), lenalidomide, or lenalidomide plus IL-2.

FIG. 6B is a line graph comparing the granzyme B production (pg/ml) in cells treated with lenalidomide alone, lenalidomide plus IL-2, or lenalidomide, IL-2, and INH II (proteinase inhibitor II).

FIG. 6C is a line graph comparing perforin production (pg/ml) in cells treated with lenalidomide alone, lenalidomide plus IL-2, or lenalidomide, IL-2, and INH II.

FIG. 6D is a line graph comparing the percentage of cytotoxicity in cells treated with lenalidomide alone, or in addition to Rituximab, Rituximab and granzyme B INH II, or Rituximab plus anti-Fas-L antibody.

FIG. 6E is a line graph comparing the percentage of cytotoxicity in cells treated with lenalidomide alone, or in addition to Rituximab, Rituximab and MSIgG (mouse IgG), or Rituximab plus anti-Fas-L antibody. Fas-L indicates Fas Ligand.

FIG. 7 is a panel of bar graphs showing the quantitation of an immunoblot comparison of cells treated with either IL-12 alone, IgG alone, IL-12 plus IgG, or IL-12 plus IgG plus lenalidomide (at 0.1 μM to 10 μM).

FIG. 7A: The percentage of SRC homology-2 containing inositol 5′phosphatase 1 (pSHIP-1) expression (normalized to total pSHIP-1) is measured.

FIG. 7B: the percentage of phospholipase C-γ2 (pPLC-γ2) expression (normalized to total pPLC-γ2) is measured.

FIG. 7C: The percentage of phosphorylated extracellular signal-related kinase (pERK ½) expression (normalized to total pERK ½) is measured.

FIG. 8 is an illustration of NK cell cytotoxicity against various antibody-coated tumor cell lines in the presence of an immunomodulatory compound:

FIG. 8A is an illustration of NK cell cytotoxicity against Trastuzumab-coated SK-BR-3 cells in the presence of lenalidomide and IL-2.

FIG. 8B is an illustration of NK cell cytotoxicity against Rituximab-coated Raji cells in the presence of lenalidomide and IL-2.

FIG. 8C is an illustration of NK cell cytotoxicity against Cetuximab-coated HCT-116 cells in the presence of lenalidomide and IL-2.

FIG. 8D is an illustration of NK cell cytotoxicity against Trastuzumab-coated SK-BR-3 cells in the presence of lenalidomide and IL-12.

FIG. 8E is an illustration of NK cell cytotoxicity against Rituximab-coated Raji cells in the presence of lenalidomide and IL-12.

FIG. 9 is an illustration of NK cell cytotoxicity against various antibody-coated tumor cell lines in the presence of an immunomodulatory compound:

FIG. 9A is an illustration of NK cell cytotoxicity against Trastuzumab-coated SK-BR-3 cells in the presence of pomalidomide and IL-2.

FIG. 9B is an illustration of NK cell cytotoxicity against Rituximab-coated Raji cells in the presence of pomalidomide and IL-2.

FIG. 9C is an illustration of NK cell cytotoxicity against Cetuximab-coated HCT-116 cells in the presence of pomalidomide and IL-2.

FIG. 9D is an illustration of NK cell cytotoxicity against Trastuzumab-coated SK-BR-3 cells in the presence of pomalidomide and IL-12.

FIG. 9E is an illustration of NK cell cytotoxicity against Rituximab-coated Raji cells in the presence of pomalidomide and IL-12.

FIG. 10 is an illustration of the enhancement of ADCC in HCT-116 cells in the presence or absence of an immunomodulatory compound. (FIG. 10A: lenalidomide and Cetuximab; and FIG. 10B: pomalidomide and Cetuximab).

FIG. 11 illustrates the enhancement in NK cell cytokine/chemokine production in response to Trastuzumab-coated SK-BR3 cells (11A: GM-CSF; 11B: IL-8; 11C: IP-10; 11D: MCP-1; 11E: MIP-1α; 11F: MIP-1β; and 11G: RANTES).

FIG. 12 is a bar graph comparing the percentage of CD56⁺ NK cells expressing Fas-L in cells treated with DMSO alone (control), an immunomodulatory compound, an immunomodulatory compound plus IL-2, or an immunomodulatory compound plus IL-12.

FIG. 13 is a panel of bar graphs showing the quantitation of an immunoblot comparison of cells treated with either IL-12 alone, IgG alone, IL-12 plus IgG, or IL-12 plus IgG plus pomalidomide (at 0.01 μM to 10 μM).

FIG. 13A: The percentage of SRC homology-2 containing inositol 5′phosphatase 1 (pSHIP-1) expression (normalized to total pSHIP-1) is measured.

FIG. 13B: the percentage of phospholipase C-γ2 (pPLC-γ2) expression (normalized to total pPLC-γ2) is measured.

FIG. 13C: The percentage of phosphorylated extracellular signal-related kinase (pERK ½) expression (normalized to total pERK ½) is measured.

### 5. DETAILED DESCRIPTION

Immunomodulatory compounds provided herein can be effective in treating many types of cancer. For example, immunomodulatory compounds have significant activity in treating myelodysplastic syndromes, multiple myeloma, and non-Hodgkin's lymphoma (NHL). Without being limited to a particular theory, administration of immunomodulatory compounds provided herein can directly enhance interferon-gamma (IFN-γ) production via Fc-γ receptor-mediated signaling in response to immunoglobulin G (IgG). Further, without being limited by a particular theory, the immunomodulatory compounds have also been found to be an enhancer of NK- and monocyte-mediated tumor cell ADCC of a variety of antibody-treated cancer cell lines in vitro. In some embodiments, the effect may be dependent on the presence of antibody and either IL-2 or IL-12.

Thus, as shown herein, and without being limited by a theory, it was found that the combination of an immunomodulatory compound and an antibody that targets a cancer cell can be useful to target tumor-specific antigens in cancer patients. Further, without being limited by a theory, it was found that an immunomodulatory compound can be combined with IgG, such as serum IgG, to effectively treat cancer. Moreover, without being limited by a theory, it was found that an immunomodulatory compound can be combined with both an antibody and IL-2 and/or IL-12 to target cancer cells.

Immunomodulatory compounds can be effective in treating many types of diseases, such as cancer, an immunological disorder, a viral infection, a fungal infection, a protozoal infection, a bacterial infection, or other diseases. In some embodiments, the immunomodulatory compound can be a compound known as an IMiD® (Celgene Corporation). Exemplary immunomodulatory compounds are described herein elsewhere. As used herein and unless otherwise indicated, the term “immunomodulatory compound” can encompass certain small organic molecules that inhibit LPS induced monocyte TNF-α, IL-1β, IL-12, IL-6, MIP-1α, MCP-1, GM-CSF, G-CSF, and COX-2 production.

As shown herein, but without being limited by a theory, the combination of an immunomodulatory compound and other agents, such as antibodies or IgG, can be effective in targeting tumor-specific antigens in cancer patients, and can therefore be useful as a cancer treatment regimen. In some embodiment, the treatment regimen may optionally include the administration of cytokines such as IL-2 or IL-12.

In an embodiment, a method of treating cancer in a patient is provided. The method comprises administering an immunomodulatory compound in combination with immunoglobulin G (IgG). In some embodiment, the method may further comprise administering cytokines such as IL-2 or IL-12.

The immunomodulatory compound can be, for example, lenalidomide or pomalidomide. The immunomodulatory compound can be administered after administration of the IgG.

The above-mentioned immunomodulatory compound can be, for example, a compound of formula I, II, III, IV, V, VI, VII, VIII, IX, X, XI, XII, XIII, XIV, XV, XVI, XVII, XVIII or a pharmaceutically acceptable salt, solvate, or stereoisomer thereof:

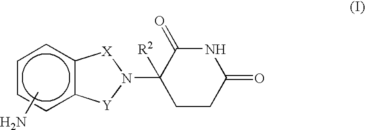

wherein:

one of X and Y is C═O, the other of X and Y is C═O or CH₂;

R² is hydrogen or lower alkyl;

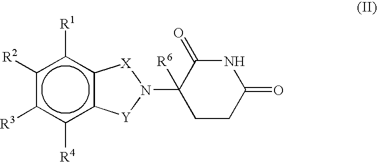

wherein:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms, or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino;

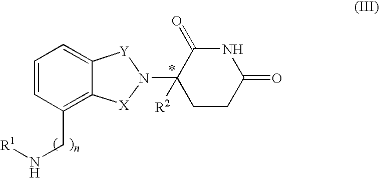

wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R³, C(S)NR³R³ or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;

n is 0 or 1; and

* is a chiral-carbon center;

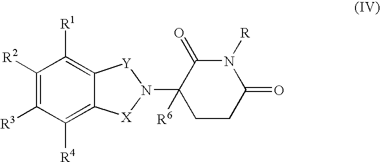

wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center;

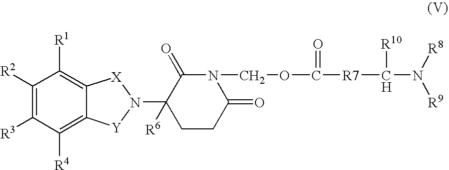

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂ X¹CH₂CH₂— in which X¹ is —O—, —S—, or —NH—; and

R¹⁰ is hydrogen, alkyl of to 8 carbon atoms, or phenyl;

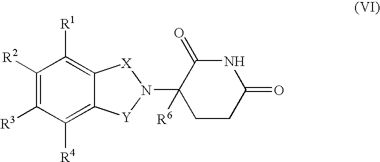

wherein:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro;

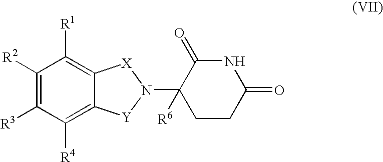

wherein:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro;

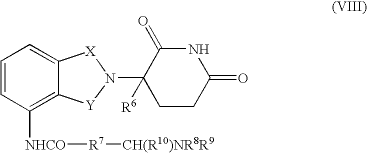

wherein:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl;

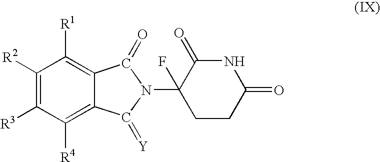

wherein:

Y is oxygen or H² and

each of R¹, R², R³, and R⁴, independently of the others, is hydrogen, halo, alkyl of 1 to 4 carbon atoms, alkoxy of 1 to 4 carbon atoms, or amino;

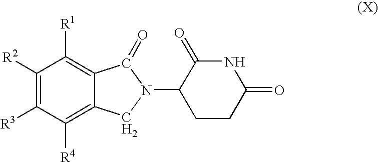

wherein:

each of R¹, R², R³, and R⁴, independently of the others, is halo, alkyl of 1 to 4 carbon atoms, or alkoxy of 1 to 4 carbon atoms;

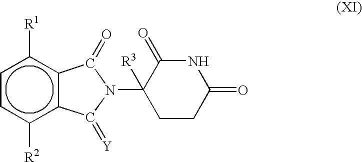

wherein:

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl;

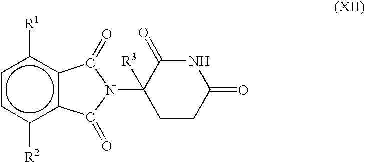

wherein:

a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and

R³ is hydrogen, alkyl of from 1 to 4 carbon atoms, or benzyl;

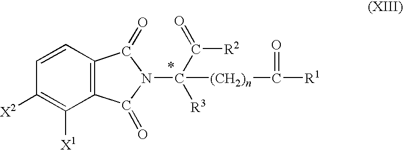

wherein:

when n is not zero and R¹ is not the same as R², C* is a center of chirality;

one of X¹ and X² is amino, nitro, alkyl of one to six carbons, or NH—Z, and the other of X¹ or X² is hydrogen;

each of R¹ and R² independent of the other, is hydroxy or NH—Z; R³ is hydrogen, alkyl of one to six carbons, halo, or haloalkyl;

Z is hydrogen, aryl, alkyl of one to six carbons, formyl, or acyl of one to six carbons; and

n has a value of 0, 1, or 2;

provided that if X¹ is amino, and n is 1 or 2, then R¹ and R² are not both hydroxy;

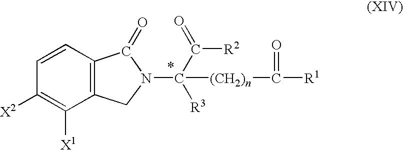

wherein:

when n is not zero and R¹ is not R², C* is a center of chirality;

one of X¹ and X² is amino, nitro, alkyl of one to six carbons, or NH—Z, and the other of X¹ or X² is hydrogen;

each of R¹ and R² independent of the other, is hydroxy or NH—Z; R³ is alkyl of one to six carbons, halo, or hydrogen;

Z is hydrogen, aryl or an alkyl or acyl of one to six carbons; and

n has a value of 0, 1, or 2;

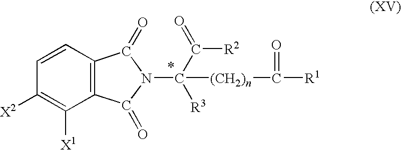

wherein:

when n is not zero and R¹ is not R², C* is a center of chirality;

one of X¹ and X² is amino, nitro, alkyl of one to six carbons, or NH—Z, and the other of X¹ or X² is hydrogen;

each of R¹ and R² independent of the other, is hydroxy or NH—Z; R³ is alkyl of one to six carbons, halo, or hydrogen;

Z is hydrogen, aryl, or an alkyl or acyl of one to six carbons; and

n has a value of 0, 1, or 2;

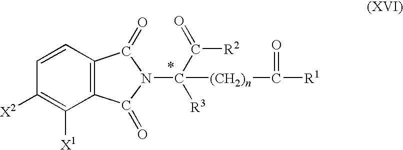

wherein:

one of X¹ and X² is nitro, or NH—Z, and the other of X¹ or X² is hydrogen;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons;

n has a value of 0, 1, or 2; and

if —COR² and —(CH₂)ₙCOR¹ are different, C* is a center of chirality;

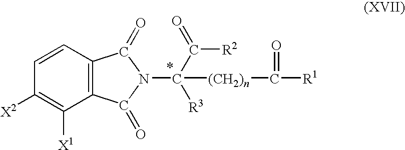

wherein:

one of X¹ and X² is alkyl of one to six carbons;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons;

n has a value of 0, 1, or 2; and

if —COR² and —(CH₂)ₙCOR¹ are different, C* is a center of chirality;

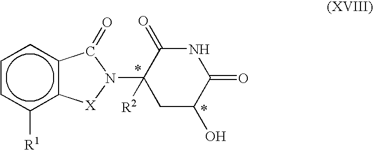

wherein:

the * carbons are centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴, wherein

R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

In some embodiments, the immunomodulatory compound can be administered from about 24 hours to about 2 weeks after the IgG administration. In additional embodiments, the immunomodulatory compound can be administered from about 12 hours to about 1 week after the administration of the IgG. The immunomodulatory compound can be administered from about 1 hour to about 2 days after the IgG administration.

In some embodiments, the IgG can be human serum IgG, such as, for example, purified human serum IgG. In additional embodiments, the IgG can be a monoclonal or polyclonal antibody. In yet additional embodiments, the IgG can be a chimeric antibody. In other embodiments, the cancer can be, for example, NHL or CLL.

In an embodiment, provided herein is a method of treating cancer in a subject comprising administering an immunomodulatory compound in combination with the anti-CD20 antibody Rituximab (Rituxan®). Immunomodulatory compound may be administered prior to, together with, or subsequent to the administration of Rituximab.

In one embodiment, the method comprises administering an immunomodulatory compound to the subject prior to administration of Rituximab. The immunomodulatory compound can be administered, for example, from about 30 minutes to about 2 weeks prior to the administration of the Rituximab. The immunomodulatory compound can be administered, for example, from about 30 minutes to about 1 week prior to the administration of the Rituximab. The immunomodulatory compound can be administered, for example, from about 1 hour to about 2 days prior to Rituximab administration. The immunomodulatory compound can be, for example, lenalidomide or pomalidomide. The method can also comprise administering IL-2 or IL-12 to the subject. The method can also comprise administering human serum IgG to the subject.

In some embodiments, the immunomodulatory compound combination therapy provides a synergistic or additive improvement in therapeutic efficacy relative to the individual therapeutic agents or compounds when administered alone. For example, the combination of an immunomodulatory compound with the anti-CD20 antibody Rituximab provides a synergistic or additive effect.

In another embodiment, provided herein is a method of treating cancer in a subject comprising administering an immunomodulatory compound in combination with the anti-HER2 antibody Trastuzumab (Herceptin®). Immunomodulatory compound may be administered prior to, together with, or subsequent to the administration of Trastuzumab.

In one embodiment, the method comprises administering an immunomodulatory compound to the subject prior to administration of Trastuzumab. The immunomodulatory compound can be administered, for example, from about 30 minutes to about 2 weeks prior to the administration of the Trastuzumab. The immunomodulatory compound can be administered, for example, from about 30 minutes to about 1 week prior to the administration of the Trastuzumab. The immunomodulatory compound can be administered, for example, from about 1 hour to about 2 days prior to Trastuzumab administration. The immunomodulatory compound can be, for example, lenalidomide or pomalidomide. The method can also comprise administering IL-2 or IL-12 to the subject. The method can also comprise administering human serum IgG to the subject.

In some embodiments, the immunomodulatory compound combination therapy provides a synergistic or additive improvement in therapeutic efficacy relative to the individual therapeutic agents or compounds when administered alone. For example, the combination of an immunomodulatory compound with the anti-HER-2 antibody Trastuzumab provides a synergistic or additive effect.

In another embodiment, provided herein is a method of treating cancer in a subject comprising administering an immunomodulatory compound in combination with the anti-EGFR antibody Cetuximab (Erbitux®). Immunomodulatory compound may be administered prior to, together with, or subsequent to the administration of Cetuximab.

In one embodiment, the method comprises administering an immunomodulatory compound to the subject prior to administration of Cetuximab. The immunomodulatory compound can be administered, for example, from about 30 minutes to about 2 weeks prior to the administration of the Cetuximab. The immunomodulatory compound can be administered, for example, from about 30 minutes to about 1 week prior to the administration of the Cetuximab. The immunomodulatory compound can be administered, for example, from about 1 hour to about 2 days prior to Cetuximab administration. The immunomodulatory compound can be, for example, lenalidomide or pomalidomide. The method can also comprise administering IL-2 or IL-12 to the subject. The method can also comprise administering human serum IgG to the subject.

In some embodiments, the immunomodulatory compound combination therapy provides a synergistic or additive improvement in therapeutic efficacy relative to the individual therapeutic agents or compounds when administered alone. For example, the combination of an immunomodulatory compound with the anti-EGFR antibody Cetuximab provides a synergistic or additive effect.

In some embodiments, an immunomodulatory compound and IgG can be administered optionally in combination with the cytokine IL-2 or IL-12. In other embodiments, an immunomodulatory compound and antibodies, such as Rituximab, Trastuzumab, or Cetuximab, can be administered optionally in combination with IL-2 or IL-12. The IL-2 or IL-12 can be full length, or can be a partial length polypeptide. The IL-2 or IL-12 can be purified, and can be obtained commercially, if desired.

The antibodies, IL-2, or IL-12 that can be added in combination with the immunomodulatory compound can also encompass mutants, derivatives (e.g., modified forms), or truncated forms of naturally occurring proteins that exhibit, in vivo, at least some of the pharmacological activity of the proteins upon which they are based. Examples of mutants include, but are not limited to, proteins that have one or more amino acid residues that differ from the corresponding residues in the naturally occurring forms of the proteins. Also encompassed by the term “mutants” are proteins that lack carbohydrate moieties normally present in their naturally occurring forms (e.g., nonglycosylated forms). Examples of derivatives include, but are not limited to, pegylated derivatives and fusion proteins.

5.1 Definitions

As used herein, and unless otherwise specified, the terms “treat,” “treating” and “treatment” refer to an action that occurs while a patient is suffering from the specified cancer, which reduces the severity of the cancer, or retards or slows the progression of the cancer.

As used herein, unless otherwise specified, the terms “prevent,” “preventing” and “prevention” refer to an action that occurs before a patient begins to suffer from the specified cancer, which inhibits or reduces the severity of the cancer.

As used herein, and unless otherwise indicated, the terms “manage,” “managing” and “management” encompass preventing the recurrence of the specified cancer in a patient who has already suffered from the cancer, and/or lengthening the time that a patient who has suffered from the cancer remains in remission. The terms encompass modulating the threshold, development and/or duration of the cancer, or changing the way that a patient responds to the cancer.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent.

As used herein, and unless otherwise specified, the term “prophylactically effective amount” of a composition is an amount sufficient to prevent cancer, or one or more symptoms associated with cancer, or prevent its recurrence. The term “prophylactically effective amount” can encompass an amount that improves overall prophylaxis or enhances the prophylactic efficacy of another prophylactic agent.

An improvement in the cancer or cancer-related disease can be characterized as a complete or partial response. By “complete response” is intended an absence of clinically detectable disease with normalization of any previously abnormal radiographic studies, bone marrow, and cerebrospinal fluid (CSF) or abnormal monoclonal protein measurements. By “partial response” is generally intended at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The terms “treatment” and “prevention” contemplate both a complete and a partial response.

“Tumor,” as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal tissue growth. Thus, “neoplastic cells” include malignant and benign cells having dysregulated or unregulated cell growth.

The terms “cancer” and “cancerous” refer to or describe the physiological condition in mammals that is typically characterized by unregulated cell growth. Examples of cancer include, but are not limited to, lymphoma and leukemia, and solid tumors. By “B cell-related cancer” or “cancer of B-cell lineage” is intended any type of cancer in which the dysregulated or unregulated cell growth is associated with B cells.

The term “antibody” is used herein in the broadest sense and covers fully assembled antibodies, antibody fragments which retain the ability to specifically bind to the antigen (e.g., Fab, F(ab′)2, Fv, and other fragments), single chain antibodies, diabodies, antibody chimeras, hybrid antibodies, bispecific antibodies, humanized antibodies, and the like), and recombinant peptides comprising the forgoing. The term “antibody” covers both polyclonal and monoclonal antibodies.

5.2 Compounds

5.2.1 Immunomodulatory Compounds

Any suitable immunomodulatory compounds can be used in the combination therapy methods described herein. Exemplary immunomodulatory compounds that can be administered include but are not limited to N-{[2-(2,6-dioxo(3-piperidyl)-1,3-dioxoisoindolin-4-yl]methyl}cyclopropyl-carboxamide; 3-[2-(2,6-dioxo-piperidin-3-yl)-1,3-dioxo-2,3-dihydro-1H-isoindol-4-ylmethyl]-1,1-dimethyl-urea; (−)-3-(3,4-Dimethoxy-phenyl)-3-(1-oxo-1,3-dihydro-isoindol-2-yl)-propionamide; (+)-3-(3,4-Dimethoxy-phenyl)-3-(1-oxo-1,3-dihydro-isoindol-2-yl)-propionamide; (−)-{2-[1-(3-ethoxy-4-methoxyphenyl)-2-methylsulfonylethyl]-4-acetylaminoisoindoline-1,3-dione}; (+)-{2-[1-(3-ethoxy-4-methoxyphenyl)-2-methylsulfonylethyl]-4-acetylaminoisoindoline-1,3-dione}; Difluoro-methoxy SelCIDs; 1-phthalimido-1-(3,4-diethoxyphenyl)ethane; 3-(3,4-dimethoxyphenyl)-3-(3,5-dimethoxyphenyl)acrylo nitrile; 1-oxo-2-(2,6-dioxopiperidin-3-yl)-4-aminoisoindoline; 1,3-dioxo-2-(2,6-dioxopiperidin-3-yl)-4-aminoisoindoline; 4-amino-2-(3-methyl-2,6-dioxo-piperidine-3-yl)-isoindole-1,3-dione; 3-(3-acetoamidophthalimido)-3-(3-ethoxy-4-methoxyphenyl)-N-hydroxypropionamide; 1-oxo-2-(2,6-dioxopiperidin-3-yl)-4-methylisoindoline; Cyclopropyl-N-{2-[(1S)-1-(3-ethoxy-4-methoxyphenyl)-2-(methylsulfonyl)ethyl]-3-oxoisoindoline-4-yl}carboxamide; Substituted 2-(3-hydroxy-2,6-dioxopiperidin-5-yl)isoindoline; N-[2-(2,6-Dioxo-piperidin-3-yl)-1,3-dioxo-2,3-dihydro-1H-isoindol-5-ylmethyl]-4-trifluoromethoxybenzamide; (S)-4-chloro-N-((2-(3-methyl-2,6-dioxopiperidin-3-yl)-1,3-dioxoisoindolin-5-yl)methyl)benzamide; Pyridine-2-carboxylic acid [2-[(3S)-3-methyl-2,6-dioxo-piperidin-3-yl]-1,3-dioxo-2,3-dihydro-1H-isoindol-5-ylmethyl]-amide; (S)—N-((2-(3-methyl-2,6-dioxopiperidin-3-yl)-1,3-dioxoisoindolin-5-yl)methyl)-4-(trifluoromethyl)benzamide; 3-(2,5-dimethyl-4-oxo-4H-quinazolin-3-yl)-piperidine-2,6-dione, and the like.

Without being limited by theory, immunomodulatory compounds disclosed herein may be potent co-stimulators of T cells and increase cell proliferation dramatically in a dose dependent manner. Immunomodulatory compounds disclosed herein may also have a greater co-stimulatory effect on the CD8+ T cell subset than on the CD4+ T cell subset. In addition, the compounds may have anti-inflammatory properties against myeloid cell responses, yet efficiently co-stimulate T cells to produce greater amounts of IL-2, IFN-γ, and to enhance T cell proliferation and CD8+ T cell cytotoxic activity. Further, without being limited by a particular theory, immunomodulatory compounds disclosed herein may be capable of acting both indirectly through cytokine activation and directly on Natural Killer (“NK”) cells and Natural Killer T (“NKT”) cells, and increase the NK cells' ability to produce beneficial cytokines such as, but not limited to, IFN-γ, and to enhance NK and NKT cell cytotoxic activity.

Specific examples of immunomodulatory compounds include cyano and carboxy derivatives of substituted styrenes such as those disclosed in U.S. Pat. No. 5,929,117; 1-oxo-2-(2,6-dioxo-3-fluoropiperidin-3yl)isoindolines and 1,3-dioxo-2-(2,6-dioxo-3-fluoropiperidine-3-yl)isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl)isoindolines (e.g., 4-methyl derivatives of thalidomide), substituted 2-(2,6-dioxopiperidin-3-yl)phthalimides and substituted 2-(2,6-dioxopiperidin-3-yl)-1-oxoisoindoles including, but not limited to, those disclosed in U.S. Pat. Nos. 5,635,517, 6,281,230, 6,316,471, 6,403,613, 6,476,052 and 6,555,554; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; and isoindole-imide compounds such as those described in U.S. patent publication no. 2003/0045552 published on Mar. 6, 2003, U.S. Pat. No. 7,091,353, and International Application No. PCT/US01/50401 (International Publication No. WO 02/059106). US patent publication no. 2006/0205787 describes 4-amino-2-(3-methyl-2,6-dioxopiperidin-3-yl)-isoindole-1,3-dione compositions. US patent publication no. 2007/0049618 describes isoindole-imide compounds. The entireties of each of the patents and patent applications identified herein are incorporated by reference. In one embodiment, immunomodulatory compounds do not include thalidomide.

Various immunomodulatory compounds disclosed herein contain one or more chiral centers, and can exist as racemic mixtures of enantiomers or mixtures of diastereomers. Provided herein is the use of stereomerically pure forms of such compounds, as well as the use of mixtures of those forms. For example, mixtures comprising equal or unequal amounts of the enantiomers of a particular immunomodulatory compounds may be used. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972), each of which is incorporated by reference herein in its entirety.

Immunomodulatory compounds provided herein include, but are not limited to, 1-oxo-and 1,3 dioxo-2-(2,6-dioxopiperidin-3-yl)isoindolines substituted with amino in the benzo ring as described in U.S. Pat. No. 5,635,517 which is incorporated herein by reference.

These compounds have the structure I

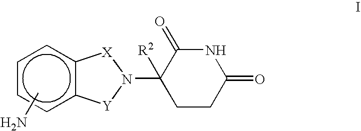

in which one of X and Y is C═O, the other of X and Y is C═O or CH₂, and R² is hydrogen or lower alkyl, in particular methyl. Specific immunomodulatory compounds include, but are not limited to:

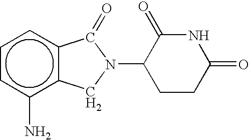

1-oxo-2-(2,6-dioxopiperidin-3-yl)-4-aminoisoindoline;

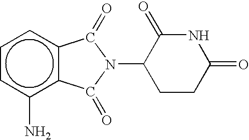

1,3-dioxo-2-(2,6-dioxopiperidin-3-yl)-4-aminoisoindoline; and

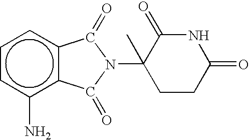

1,3-dioxo-2-(3-methyl-2,6-dioxopiperidin-3-yl)-4-aminoisoindole, and optically pure isomers thereof.

The compounds can be obtained via standard, synthetic methods (see e.g., U.S. Pat. No. 5,635,517, incorporated herein by reference). The compounds are also available from Celgene Corporation, Warren, N.J.

Other specific immunomodulatory compounds belong to a class of substituted 2-(2,6-dioxopiperidin-3-yl)phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula:

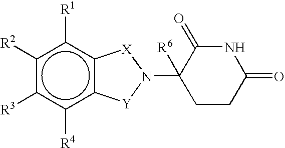

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas:

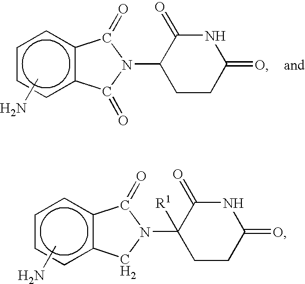

wherein R¹ is hydrogen or methyl. In a separate embodiment, provided herein is the use of enantiomerically pure forms (e.g. optically pure (R) or (S) enantiomers) of these compounds.

Still other specific immunomodulatory compounds disclosed herein belong to a class of isoindole-imides disclosed in U.S. Pat. No. 7,091,353, U.S. Patent Publication No. 2003/0045552, and International Application No. PCT/US01/50401 (International Publication No. WO 02/059106), each of which are incorporated herein by reference. Representative compounds are of formula II:

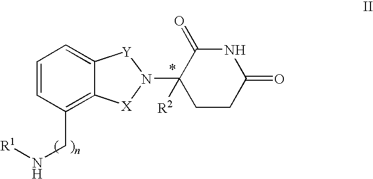

and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)2, (C₁-C₈)alkyl-OR⁵, (C₁-C₈)alkyl-C(O)OR⁵, C(O)NHR³, C(S)NHR³, C(O)NR³R^{3′}, C(S)NR³R³′ or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)2, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;
R² is H or (C₁-C₈)alkyl; and
R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)2; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

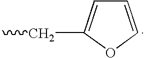

In another embodiment of the compounds of formula II, R¹ is

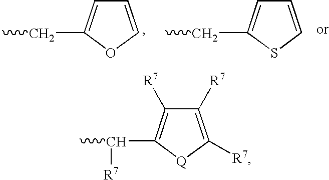

wherein Q is O or S, and each occurrence of R⁷ is independently H,(C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)2, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl]methyl}(benzylamino)carboxamide.

Still other specific immunomodulatory compounds disclosed herein belong to a class of isoindole-imides disclosed in U.S. Pat. No. 6,555,554, International Publication No. WO 98/54170, and U.S. Pat. No. 6,395,754, each of which is incorporated herein by reference. Representative compounds are of formula III:

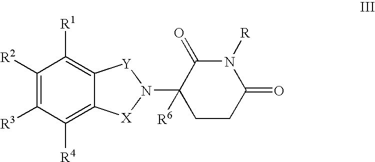

and pharmaceutically acceptable salts, hydrates, solvates, clathrates, enantiomers, diastereomers, racemates, and mixtures of stereoisomers thereof, wherein:
one of X and Y is C═O and the other is CH₂ or C═O;
R is H or CH₂OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CnH2n)- in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—;
R¹⁰ is hydrogen, alkyl of to 8 carbon atoms, or phenyl; and
* represents a chiral-carbon center.

Other representative compounds are of formula:

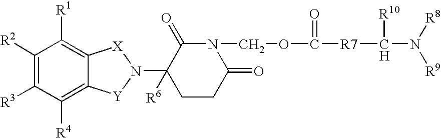

wherein:
one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CnH2n)- in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂ X¹CH₂CH₂— in which X¹ is —O—, —S—, or —NH—; and

R¹⁰ is hydrogen, alkyl of to 8 carbon atoms, or phenyl.

Other representative compounds are of formula:

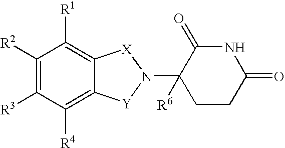

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

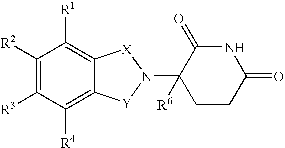

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

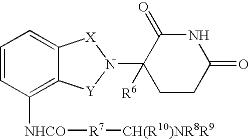

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CnH2n)- in which n has a value of 0 to 4; each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Other specific immunomodulatory compounds are 1-oxo-2-(2,6-dioxo-3-fluoropiperidin-3yl)isoindolines and 1,3-dioxo-2-(2,6-dioxo-3-fluoropiperidine-3-yl)isoindolines such as those described in U.S. Pat. Nos. 5,874,448 and 5,955,476, each of which is incorporated herein by reference. Representative compounds are of formula:

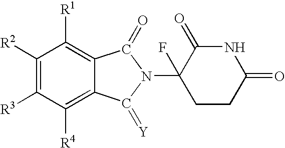

wherein:
Y is oxygen or H₂ and
each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds are the tetra substituted 2-(2,6-dioxopiperdin-3-yl)-1-oxoisoindolines described in U.S. Pat. No. 5,798,368, which is incorporated herein by reference. Representative compounds are of formula:

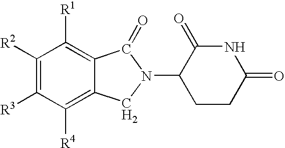

wherein each of R¹, R², R³, and R⁴, independently of the others, is halo, alkyl of 1 to 4 carbon atoms, or alkoxy of 1 to 4 carbon atoms.

Other specific immunomodulatory compounds are 1-oxo and 1,3-dioxo-2-(2,6-dioxopiperidin-3-yl)isoindolines disclosed in U.S. Pat. No. 6,403,613, which is incorporated herein by reference. Representative compounds are of formula:

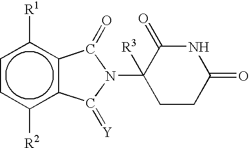

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

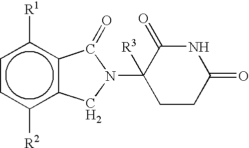

wherein
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

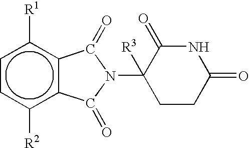

wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl.

Other specific immunomodulatory compounds disclosed herein are 1-oxo and 1,3-dioxoisoindolines substituted in the 4- or 5-position of the indoline ring described in U.S. Pat. No. 6,380,239 and U.S. Pat. No. 7,244,759, both of which are incorporated herein by reference. Representative compounds are of formula:

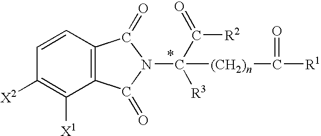

in which the carbon atom designated C* constitutes a center of chirality (when n is not zero and R¹ is not the same as R²); one of X¹ and X² is amino, nitro, alkyl of one to six carbons, or NH—Z, and the other of X¹ or X² is hydrogen; each of R¹ and R² independent of the other, is hydroxy or NH—Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula:

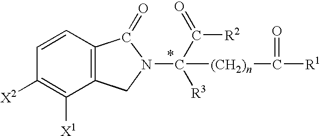

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

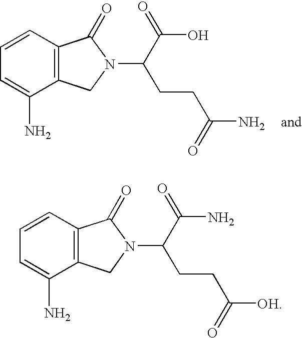

Other representative compounds are of formula:

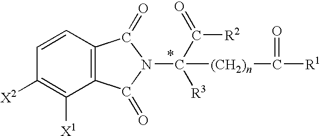

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptable salts, solvate, prodrugs, and stereoisomers thereof:

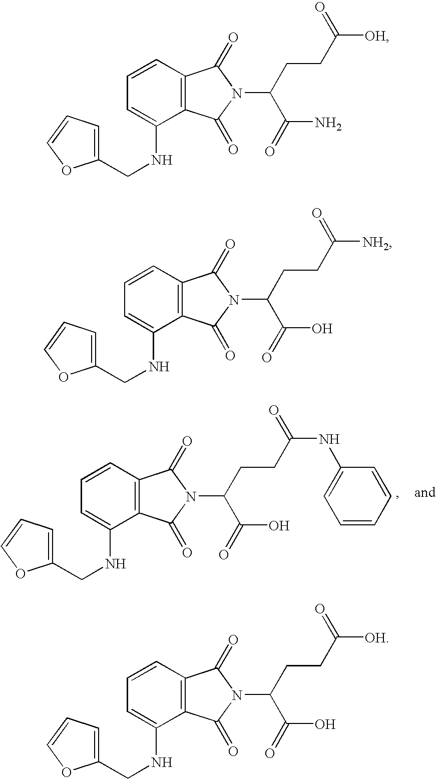

Other specific examples of the compounds are of formula:

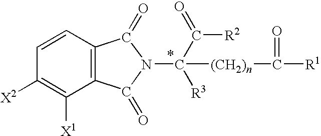

wherein:

one of X¹ and X² is nitro, or NH—Z, and the other of X¹ or X² is hydrogen;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Other representative compounds are of formula:

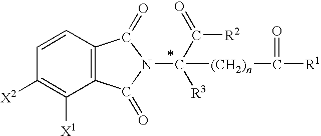

wherein:

one of X¹ and X² is alkyl of one to six carbons;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds are isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

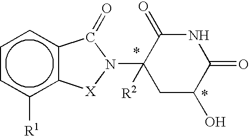

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

All of the compounds described can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques. Additional information on immunomodulatory compounds, their preparation, and use can be found, for example, in U.S. Patent Application Publication Nos. US20060188475, US20060205787, and US20070049618, each of which is incorporated by reference herein in its entirety.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

5.2.2 Antibodies

As mentioned above, an immunomodulatory compound can be administered in combination with an antibody to effectively treat cancer. Many types of antibodies can be used. In some embodiments, the antibody can be a monoclonal antibody. A monoclonal antibody (mAb), as used herein, generally refers to an antibody obtained from a substantially homogeneous population of antibodies, i.e., the individual antibodies comprising the population are identical except for possible naturally occurring mutations that can be present in minor amounts. In other embodiments, the antibody can be a polyclonal antibody.

In contrast to polyclonal antibody preparations, which typically include different antibodies directed against different antigenic determinants (epitopes), each monoclonal antibody is generally directed against a single determinant (epitope) on the antigen. An epitope is generally the portion of an antigenic molecule to which an antibody is produced and to which the antibody can bind. Epitopes can comprise linear amino acid residues (i.e., residues within the epitope are arranged sequentially one after another in a linear fashion), non-linear amino acid residues (referred to herein as “non-linear epitopes”; these epitopes are not arranged sequentially), or both linear and non-linear amino acid residues. A monoclonal antibody suitable for use in the methods provided herein can be capable of specifically binding to an epitope on a human antigen expressed on the surface of a human cell, i.e. an epitope that is exposed to the exterior of the cell.

When the composition comprises a monoclonal antibody, any suitable method for obtaining monoclonal antibodies can be used. Monoclonal antibodies can be made by the hybridoma method first described by Kohler et al. (1975) Nature 256:495, incorporated herein by reference, or can be made by recombinant DNA methods. Monoclonal antibodies can also be isolated from antibody phage libraries generated using the techniques described in, for example, McCafferty et al. (1990) Nature 348:552-554 (1990), which is incorporated by reference herein in its entirety. Further, Clackson et al. (1991) Nature 352:624-628 and Marks et al. (1991) J Mol. Biol. 222:581-597, each of which is incorporated by reference herein in its entirety, describe the isolation of murine and human antibodies, respectively, using phage libraries. Methods of producing high affinity (nM range) human antibodies by chain shuffling (Marks et al. (1992) Bio/Technology 10:779-783, which is incorporated by reference herein in its entirety), as well as combinatorial infection and in vivo recombination as a strategy for constructing very large phage libraries (Waterhouse et al. (1993) Nucleic. Acids Res. 21:2265-2266, which is incorporated by reference herein in its entirety) can be used. Alternatively, the monoclonal antibody can be obtained commercially.

In some embodiments, the antibody is IgG. In some embodiments, the IgG is serum IgG, and in particular, human serum IgG. In some embodiments, the IgG is IgG1. Human IgG, for example, can be prepared from a patient, or can be obtained from commercial sources. IgG can be isolated from blood using standard protein A-agarose and/or DEAE-chromatography. The IgG can then be dialyzed to remove excess salts, and can then be lyophilized to increase protein concentration. The IgG can also be filter sterilized. Purity of the IgG can be determined, for example, by SDS-PAGE or by other means known in the art.

In other embodiments, the antibody can be an anti-CD20 antibody. In one embodiment, the anti-CD20 antibody is Rituximab. In other embodiments, the antibody can be an anti-HER-2 antibody. In one embodiment, the anti-HER-2 antibody is Trastuzumab. In other embodiments, the antibody can be an anti-EGFR antibody. In one embodiment, the anti-EGFR antibody is Cetuximab.

5.3 Methods of Administration

Methods provided herein comprise administering one or more immunomodulatory compounds, or a pharmaceutically acceptable salt, solvate, stereoisomer, or prodrug thereof, in combination with an antibody (such as IgG or other antibodies described herein) to a patient (e.g., a human) suffering, or likely to suffer, from a cancer-related disease or disorder. In some embodiments, the methods may also comprise additionally administering IL-2 or IL-12 to the patient.

Any of the components of the composition can be administered together or separately. Each of the components can be administered by any suitable means. In some embodiments, at least a part of the formulation is administered using intravenous administration. In some embodiments, intravenous administration can occur by infusion over a period of about less than 1 hour to about 10 hours (less than 1, 2, 3, 4, 5, 6, 7, 8, 9, or 10 hours). Subsequent infusions can be administered over a period of about less than 1 to about 6 hours, including, for example, about 1 to about 4 hours, about 1 to about 3 hours, or about 1 to about 2 hours or less than an hour. Alternatively, a dose can be administered subcutaneously or by other means.

In some embodiments, at least a part of the formulation is administered orally. Pharmaceutical compositions that are suitable for oral administration can be presented as in several types of forms, such as, but are not limited to, tablets, caplets, capsules, and liquids. In some embodiments, oral administration of a component of the composition can occur prior to, after, or during the administration of the other components. For example, oral administration of at least one of the components can occur 2 weeks, 1 week, 3 days, one day, 12 hours, 1 hour, or 30 minutes prior to or after administration of the other components of the composition. Oral administration of at least one component of the composition can occur several times per day, daily, once every other day, once weekly, and the like.

A single dose of the immunomodulatory compound to be administered can be, for example, in the range from about 0.3 mg/kg of patient body weight to about 50 mg/kg, from about 0.1 mg/kg to about 40 mg/kg, from about 0.01 mg/kg to about 30 mg/kg, from about 0.1 mg/kg to about 30 mg/kg, from about 0.5 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg.

Thus, for example, the immunomodulatory compound dose can be 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or other such doses falling within the range of about 0.3 mg/kg to about 50 mg/kg.

Similarly, a single dose of the antibody to be administered can be, for example, in the range from about 0.3 mg/kg to about 50 mg/kg, from about 0.1 mg/kg to about 40 mg/kg, from about 0.01 mg/kg to about 30 mg/kg, from about 0.1 mg/kg to about 30 mg/kg, from about 0.5 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg.

Thus, for example, the antibody dose can be 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or other such doses falling within the range of about 0.3 mg/kg to about 50 mg/kg.

A single dose of IL-2 or IL-12 to be administered can be, for example, in the range from about 0.3 mg/kg to about 50 mg/kg, from about 0.1 mg/kg to about 40 mg/kg, from about 0.01 mg/kg to about 30 mg/kg, from about 0.1 mg/kg to about 30 mg/kg, from about 0.5 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg.

Thus, for example, the IL-2 or IL-12 can be 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or other such doses falling within the range of about 0.3 mg/kg to about 50 mg/kg.

When the immunomodulatory compound is administered in combination with IgG, such as serum IgG, the dose of serum IgG to be administered can be, for example, in the range from about 0.3 mg/kg to about 50 mg/kg, from about 0.1 mg/kg to about 40 mg/kg, from about 0.01 mg/kg to about 30 mg/kg, from about 0.1 mg/kg to about 30 mg/kg, from about 0.5 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg.

Thus, for example, the IgG dose can be 0.3 mg/kg, 0.5 mg/kg, 1 mg/kg, 1.5 mg/kg, 2 mg/kg, 2.5 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 45 mg/kg, or 50 mg/kg, or other such doses falling within the range of about 0.3 mg/kg to about 50 mg/kg.

Administration of the immunomodulatory compound “in combination with” one or more further therapeutic agents includes simultaneous (concurrent) and consecutive administration in any order. Thus, the immunomodulatory compound can be administered at the same time, or prior to, or after one or more of the compounds in the combination. For example, the composition to be administered can contain all of the ingredients in the combination. Alternatively, one or more of the compounds in the composition can be administered before or after the other compounds. In one embodiment, the immunomodulatory compound can be administered at the same time as the other components in the composition. In another embodiment, the immunomodulatory compound can be administered from about 0, 10, 30, to about 60 minutes or more after administration of at least one of the other compounds in the composition. In other embodiments, the immunomodulatory compound can be administered from about 1, 6, 12, or 24 hours to about 2 days, 4 days, 1 week, or about 2 weeks after administration of at least one of the other compounds in the composition. In one embodiment, the immunomodulatory compound is lenalidomide.

In an embodiment, the immunomodulatory compound can be administered from about 0, 10, 30, to about 60 minutes or more before administration of Rituximab, Trastuzumab, or Cetuximab. In another embodiment, the immunomodulatory compound can be administered from about 1, 6, 12, or 24 hours to about 2 days, 4 days, 1 week, or about 2 weeks before administration of Rituximab, Trastuzumab, or Cetuximab. In one embodiment, the immunomodulatory compound is lenalidomide. In another embodiment, the immunomodulatory compound is pomalidomide.

In another embodiment, an immunomodulatory compound is administered in combination with IgG. In an embodiment, an immunomodulatory compound is administered after the administration of IgG. In an embodiment, the immunomodulatory compound can be administered from about 0, 5, 10, 30, to about 45 minutes or more after administration of IgG. In another embodiment, the immunomodulatory compound can be administered from about 30 minutes, 1, 6, 12, 18 or 24 hours to about 2 days, 4 days, 1 week, or about 2 weeks after administration of IgG. In one embodiment, the immunomodulatory compound is lenalidomide. In another embodiment, the immunomodulatory compound is pomalidomide.

The immunomodulatory compound can be administered by the same route, or by a different route, than the other compound or compounds in the combination. For example, some of the components of the composition can be administered orally, while others are administered intravenously. In additional embodiments, some of the components are administered by subcutaneous injection, while other components are administered by infusion.

Many types of cancer can be treated using the combinations of immunomodulatory compounds plus other compounds as disclosed herein. Specific examples of cancer include, but are not limited to: cancers of the skin, such as melanoma; lymph node; breast; cervix; uterus; gastrointestinal tract; lung; ovary; prostate; colon; rectum; mouth; brain; head and neck; throat; testes; kidney; pancreas; bone; spleen; liver; bladder; larynx; nasal passages; and AIDS-related cancers. Methods provided herein can also be used to follow the treatment of cancers of the blood and bone marrow, such as multiple myeloma and acute and chronic leukemias, for example, lymphoblastic, myelogenous, lymphocytic, myelocytic leukemias, and myelodysplastic syndromes including but not limited to 5q minus syndrome, or myelodysplastic syndromes associated with other cytogenic abnormalities, and the like. The methods provided herein can be used for managing either primary or metastatic tumors.

Other specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma (localized melanoma, including, but not limited to, ocular melanoma), malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, leiomyoma, and the like. In a specific embodiment, the cancer is metastatic. In another embodiment, the cancer is refractory or resistant to chemotherapy or radiation.

In one embodiment, the cancer is NHL or CLL. In another embodiment, the cancer is breast, ovary, colon/colorectal, lung or bone cancer. In one specific embodiment, the antibody used is Rituximab, and the cancer is non-Hodgkin's lymphoma. In another specific embodiment, the antibody used is Trastuzumab, and the cancer is breast cancer. In another specific embodiment, the cancer is Cetuximab, and the cancer is colorectal cancer.

5.4 Pharmaceutical Compositions and Dosage Forms

The compositions of immunomodulatory compounds in combination with antibodies (such as IgG or other antibodies provided herein), optionally also comprising IL-2 or IL-12, can be formulated into desired dosage forms. For example, single or multiple unit dosage forms can be prepared.

The compositions can be formulated to be suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a cancer-related disease or disorder may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same cancer-related disease or disorder. These and other ways in which specific dosage forms will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients, or when exposed to water.

Also provided herein are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80, which is incorporated by reference herein in its entirety. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, strip packs, and the like.

Also provided herein are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, salt buffers, and the like.

The amounts and specific types of active ingredients in a dosage form may differ depending on factors such as, but not limited to, the route by which it is to be administered to patients. Thus, in some embodiments, typical dosage forms comprise an immunomodulatory compound or a pharmaceutically acceptable salt, solvate, stereoisomer, or prodrug thereof in an amount of from about 0.10 to about 250 mg. In some embodiments, dosage forms can comprise an immunomodulatory compound or a pharmaceutically acceptable salt, solvate, stereoisomer, or prodrug thereof in an amount of about 0.1, 1, 2, 5, 7.5, 10, 12.5, 15, 17.5, 20, 25, 50, 100, 150 or 200 mg. Typical dosage forms comprise the second active ingredient, such as IgG, an antibody, IL-2, or IL-12, in an amount of from about 0.1 to about 1000 mg, from about 5 to about 500 mg, from about 10 to about 350 mg, or from about 50 to about 200 mg. The specific amount of the agent will depend on the specific agent used, the type of cancer-related disease or disorder being treated or managed, and the amount(s) of an immunomodulatory compound and any optional additional active agents concurrently administered to the patient.

5.4.1 Parenteral Dosage Forms

Parenteral dosage forms can be administered to patients by various routes including, but not limited to, subcutaneous, intravenous (including bolus injection), intramuscular, intraarterial, and the like. Parenteral administration typically bypasses an individual's natural defenses against contaminants, so these dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, emulsions, and the like.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, benzyl benzoate, and the like.

Compounds that increase the solubility of one or more of the active ingredients disclosed herein can also be incorporated into the parenteral dosage forms provided herein. For example, cyclodextrin and its derivatives can be used to increase the solubility of an immunomodulatory compound and its derivatives. See, e.g., U.S. Pat. No. 5,134,127, which is incorporated herein by reference.

5.4.2 Oral Dosage Forms

One or more of the components of the composition can be administered orally, if desired. Pharmaceutical compositions that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms can be prepared by combining the active ingredients with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, coloring agents, and the like. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, disintegrating agents, and the like.

If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, lubricants, and the like. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, mixtures thereof, and the like.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, mixtures thereof, and the like. The binder or filler in pharmaceutical compositions is typically present in from about 50 to about 99 weight percent of the pharmaceutical composition or dosage form.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, mixtures thereof, and the like.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, mixtures thereof, and the like.

5.4.3 Delayed Release Dosage Forms

One or more of the active components of the combination composition can be administered by a delayed release means, if desired. Controlled release means or by delivery devices are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein. Thus, provided herein are single unit dosage forms suitable for oral administration such as, but not limited to, tablets, capsules, gelcaps, and caplets that are adapted for controlled-release.

Among the advantages of controlled-release formulations are the extended activity of the drug, reduced dosage frequency, and increased patient compliance. In addition, controlled-release formulations can be used to affect the time of onset of action or other characteristics, such as blood levels of the drug, and can thus affect the occurrence of side effects.

Most controlled-release formulations are designed to initially release an amount of the active ingredient that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.4.4 Topical and Mucosal Dosage Forms

In some embodiments, at least one of the components of the combination formulation can be administered topically or mucosally. Topical and mucosal dosage forms include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms are well known to those skilled in the pharmaceutical arts, and depend on the particular tissue to which a given pharmaceutical composition or dosage form will be applied. Typical excipients include, but are not limited to, water, acetone, ethanol, ethylene glycol, propylene glycol, butane-1,3-diol, isopropyl myristate, isopropyl palmitate, mineral oil, mixtures thereof, and the like. The excipients can form solutions, emulsions or gels, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Various salts, hydrates or solvates of the active ingredients can be used to further adjust the properties of the resulting composition.

5.4.5 Kits

In some embodiments, a kit for treating cancer is provided. A typical kit comprises the combination of a dosage form of an immunomodulatory compound, or a pharmaceutically acceptable salt, solvate, stereoisomer, or prodrug thereof, and an antibody (such as IgG or other antibodies provided herein). The kit may also contain IL-2 and/or IL-12. Kits provided herein can further comprise additional active and inactive ingredients.

Kits can further comprise devices that are used to administer the active ingredients. Examples of such devices include, but are not limited to, syringes, drip bags, patches, inhalers, and the like. Kits can also contain instruction sheets for use. The kits can be for single use, or can be designed for multiple dosage use.

Kits can further comprise cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, benzyl benzoate, and the like.

The following examples are offered for illustrative purposes only, and are not intended to limit the scope in any way.

### 6. EXAMPLES

6.1 Procedures

6.1.1 Preparation of Cells for Flow Cytometry

Cytometry was undertaken on 50 μL samples containing 1×10⁶ cells/mL in washing buffer, 2% FBS with 0.1% NaN₃ in PBS. Fluorochrome-conjugated antibodies, unconjugated antibodies, and isotype control mAb were obtained from PharMingen (San Diego, Calif.). The cells were stained with anti-CD56-PE, anti-FasL-FITC or isotype control mAb (1 μg/10⁶ cells), and analyzed by flow cytometry according to the manufacturer's instructions.

6.1.2 Preparation of NK Cells and Monocytes

NK cells and monocytes were isolated from fresh, buffy-coated, whole blood by 30-minute incubation with RossetteSep cocktail (StemCell Technologies, Inc.) followed by Ficoll-Hypaque density gradient centrifugation. CD56+ NK cells were isolated to ˜85% purity and CD14+ monocytes were isolated to ˜80% purity as determined by flow cytometry.

6.1.3 NK IgG-Induced IFN-65 Assay

To determine whether lenalidomide enhances IFN-γ production, the following assay was performed. Human IgG (Sigma), at a concentration of 100 μg/mL was coated onto 96-well, flat-bottom plates at 4° C. overnight. The unbound IgG was then washed away. NK cells were plated at 2×10⁵ cells/well, and 10 ng/mL of either interleukin-2 (IL-2) or IL-12 (R&D Systems, MN) was added. Lenalidomide (Revlimid®, Celgene Corporation, Summit, N.J.) was added to the wells in the concentration range of 0.008-5 μM. After a 48-hour incubation, the supernatants were harvested and interferon-gamma (IFN-γ), as well as granzyme B and perforin levels were measured.

6.2 Lenalidomide and IgG Incubated with IL-2, or IL-12 Enhances IFN-γ Production by NK Cells

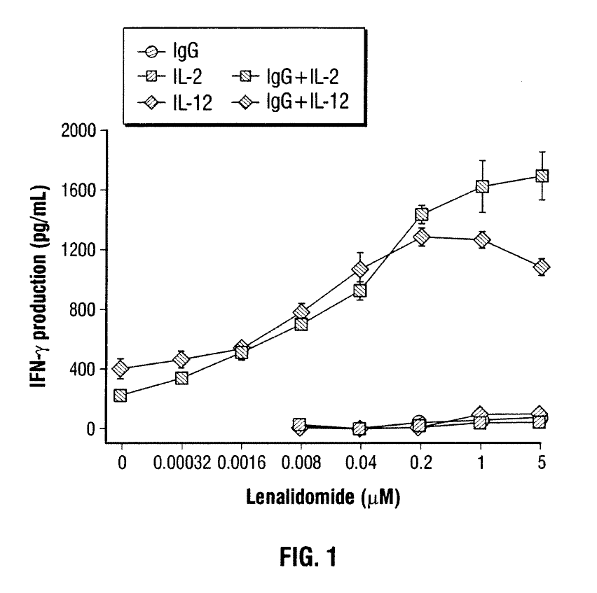

The immunomodulatory compound lenalidomide, at a concentration of (0.008-5 μM), when added to human IgG incubated with IL-2, enhanced IFN-γ production of NK cells by 3 fold, in comparison with IgG plus IL-2 alone (FIG. 1). Similarly, when lenalidomide, at a concentration of (0.008-5 μM) was added to human IgG incubated with IL-12, enhanced IFN-γ production by 8-fold respectively, in comparison with IgG plus IL-12 alone (FIG. 1).

The enhancing effect of lenalidomide was dose-dependent for both IL-2 and IL-12, and the EC₅₀ values were 0.090 μM and 0.046 μM, respectively. In contrast, when lenalidomide alone, immobilized IgG alone, or lenalidomide plus IgG was used, no IFN-γ was observed. Thus, either IL-2 or IL-12 plus immobilized IgG was required for IFN-γ production (FIG. 1).

As shown herein, administration of immunomodulatory compounds such as lenalidomide can enhance the production of IFN-γ induced by IgG and IL-2 or IL-12. Thus, lenalidomide can directly enhance IFN-γ production by NK cells. The IFN-γ production from NK cells is dependent on the presence and/or activity of IgG combined with either IL-2 or IL-12.

6.3 ADCC Assay

Administration of immunomodulatory compounds such as lenalidomide can enhance the anti-tumor activity of antibodies such as the anti-CD20 monoclonal antibody Rituximab. The following examples describe methods of examining the effect of immunomodulatory agents such as lenalidomide on the enhancement of ADCC by the antibody Rituximab.

Preparation of NK cells: Purified NK cells (5×10⁴) were seeded in 96-well U-bottom plates in 100 μL of 10% RPMI-1640 medium, supplemented with human AB+ serum. The cells were treated with lenalidomide (0.001-10 μM) plus 10 ng/mL of IL-2 or IL-12. The cells were then incubated at 37° C. overnight.

Preparation of Rituximab-coated target cancer cells: Various NHL cell lines (Namalwa, Raji, Farage, and Jeko-1) were treated with 20 μg/mL Rituximab (Rituximab, Genentech, Inc.) for 30 minutes at 37° C. Excess unbound Rituximab was then removed by a washing step.

Addition of Rituximab-coated target cells to NK cells: The Rituximab-coated target cells (5×103/100 μL/well) were added to the pretreated effector NK cells at a ratio of 10:1. The cells were then co-incubated for 4 hours at 37° C. Control groups included NK cells and tumor cells treated with medium alone, Rituximab alone, or IL-2/IL-12 alone.

Cytotoxicity analysis: NK cell cytotoxicity against the tumor cell lines was analyzed using a standard lactate dehydrogenase release assay (CytoTox 96 Non-radioactive Cytotoxicity Assay, Promega) to measure ADCC in a 50 μL aliquot of supernatant. The experimental release was corrected by subtraction of the spontaneous release of effector cells at the corresponding dilution. Spontaneous release from target cells alone was <15% of the maximum release as determined with target cells lysed in 1% Triton X-100.

Determination of effector: target ratio: Specific lysis for each effector-to-target (E:T) cell ratio was calculated using the following formula: % specific lysis=([experimental release−spontaneous release]/[maximum release−spontaneous release])×100.

To evaluate monocyte ADCC, similar methods were used to prepare the monocytes. However, the incubation time was increased to 16 hours, and IL-2 and IL-12 were omitted.

6.4 Enhancement of NK Cell Cytotoxicity in Rituximab-Coated NHL Cell Lines

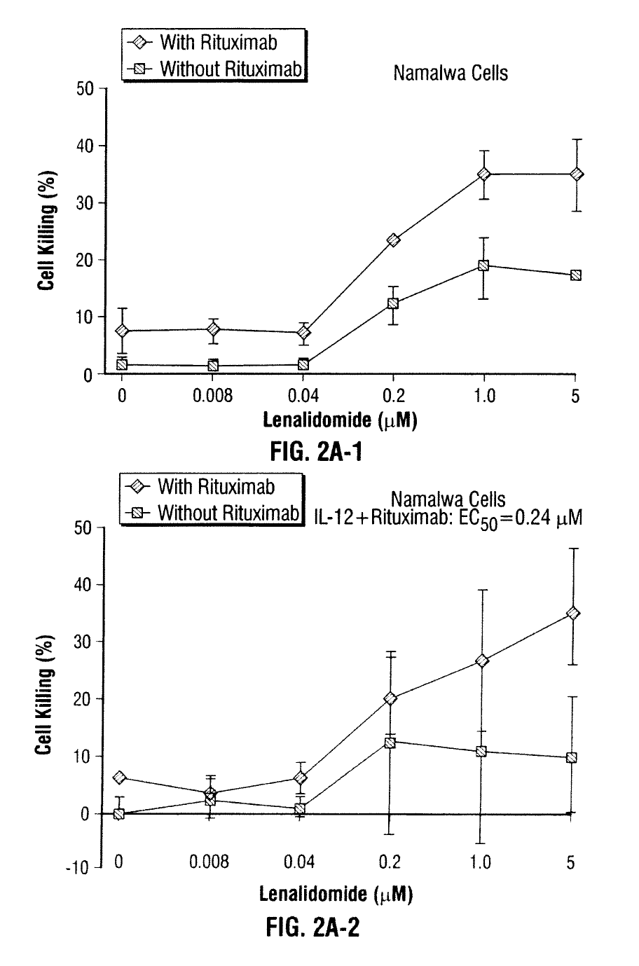

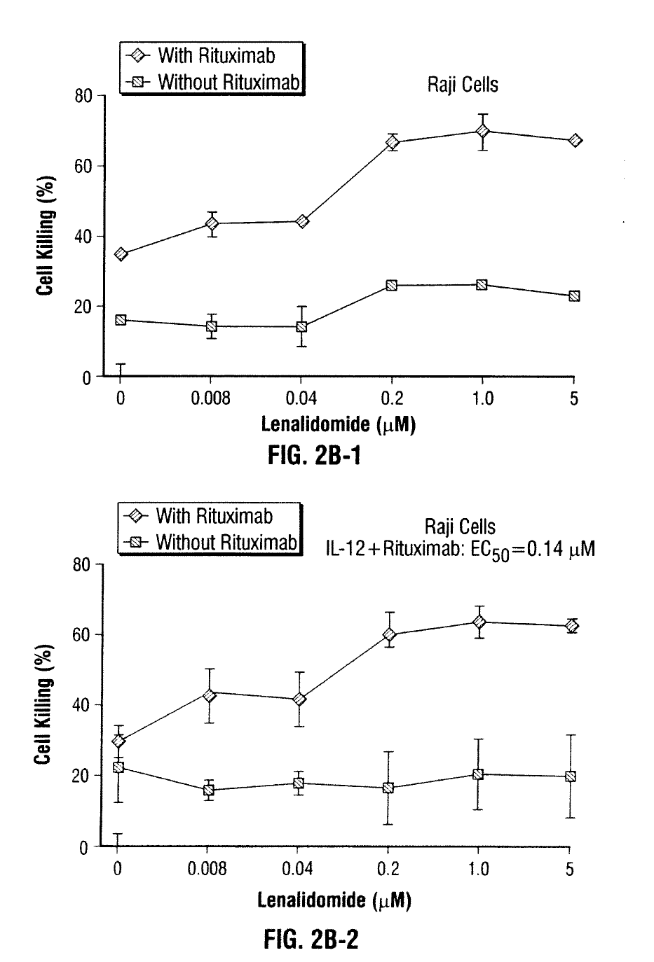

Cytotoxicity was measured following the method described in the example above. In the presence of IL-2 or IL-12, lenalidomide increased the NK cell (E:T=10:1) specific lysis of Rituximab-coated Raji and Namalwa (human Burkitt's lymphoma) NHL cells by 2- and 5-fold, respectively, compared with Rituximab-only treated cells (FIGS. 2A and 2B). The EC₅₀ for Namalwa cells was 0.19 μM for IL-2 and 0.24 μM for IL-12. For Raji cells, the EC₅₀ values were 0.10 μM and 0.14 μM, respectively.

In addition, lenalidomide dose-dependently increased the NK cell (E:T=10:1) specific lysis of Rituximab-coated human B-cell lymphoma Farage cells (FIG. 2C). Cell lysis increased 1.8-fold in the presence of IL-2 (EC₅₀=0.042 μM) and 1.6-fold in the presence of IL-12 (EC₅₀=0.033 μM) compared with Rituximab-only treated cells.

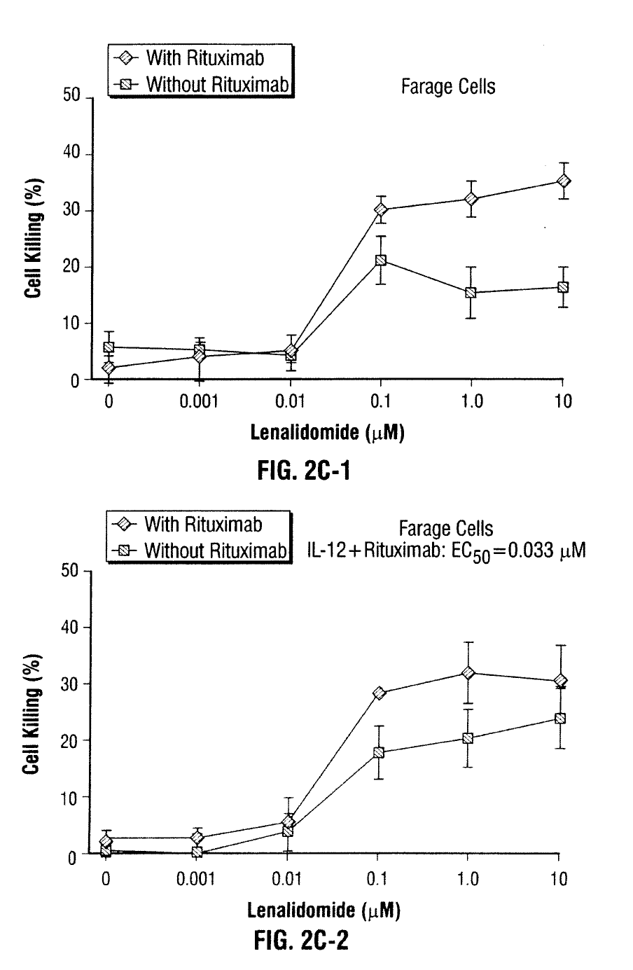

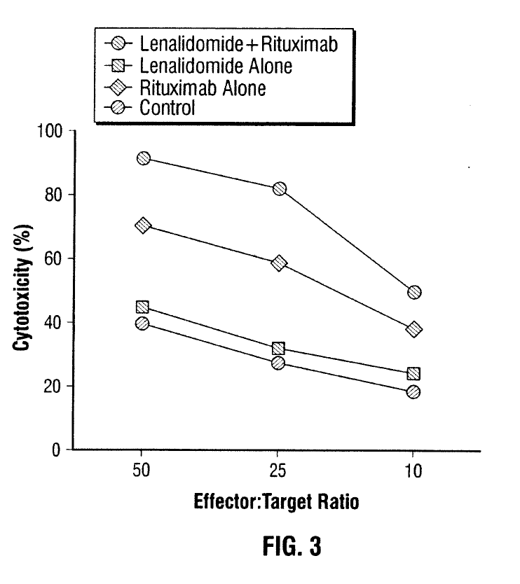

In further experiments, lenalidomide (1 μM) in the presence of IL-2 enhanced the NK cell-mediated killing of Rituximab-coated CD20+ Jeko-1 (mantle cell lymphoma) cells from 70% to 95% at the E:T ratio of 50:1 (FIG. 3). The lenalidomide effect accounted for a 1.4-fold increase in the NK cell-mediated killing activity compared with the Rituximab-only treatment and by >2-fold compared with uncoated (control) cells (FIG. 3). Lenalidomide alone accounted for a 5% increase in tumor cell killing relative to the control (background killing=40%). Rituximab-only treatment increased tumor cell killing approximately 30% above the background. However, the combined killing effects of lenalidomide and Rituximab acted synergistically, enhancing the NK cell-mediated killing of tumor cells by 55% above background.

6.5 Enhancement of Monocyte-Mediated Lysis of NHL Cells

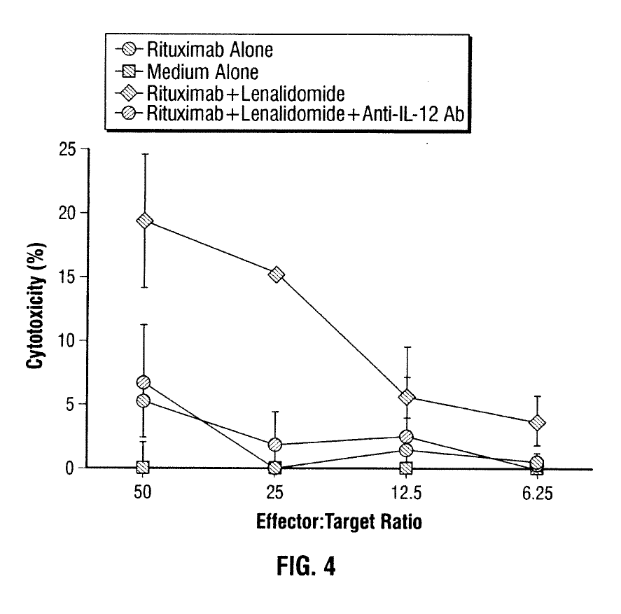

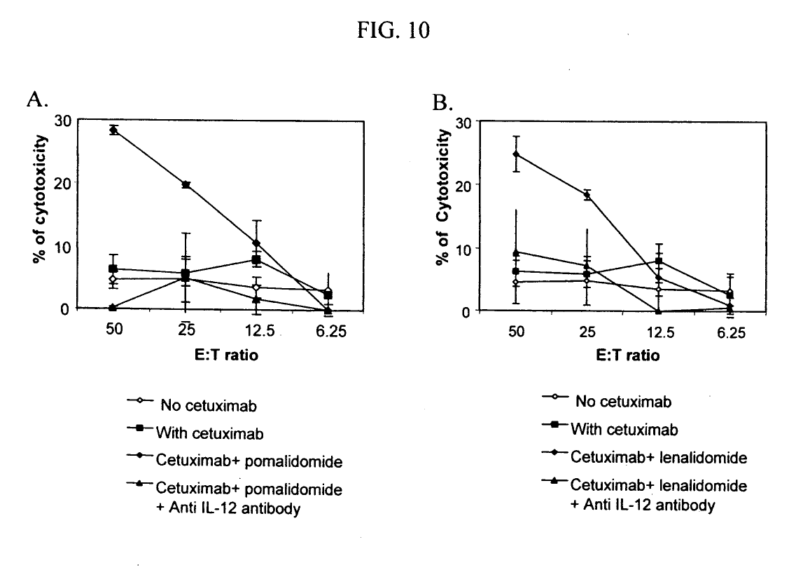

The effect of lenalidomide and pomalidomide on monocyte-mediated lysis was examined. Lealidomide and pomalidomie were found to enhance monocyte-mediated lysis of Rituximab-coated Raji cells (data not shown). The lysis of Rituximab-coated CD20+ Raji cells showed an approximate 30% maximum killing activity at an E:T ratio of 50:1. The addition of lenalidomide (1 μM) with IL-2 enhanced the monocyte-mediated killing of Rituximab-coated CD20+ Raji cells to >60% at the 50:1 E:T ratio, demonstrating a 2-fold increase in tumor cell-lysis activity. Monocyte-mediated lysis of Rituximab-coated Farage cells showed very good killing activity, from 5% up to 20% at a 50:1 ratio, in the presence of lenalidomide (at 10 μM) (FIG. 4). Similar results were observed for pomalidomide (FIG. 10B). The addition of IL-2 was not required in these assays. However, the specific lysis of Rituximab-coated Farage cells by immunomodulatory compound-pretreated monocytes was blocked by neutralizing anti-IL-12 antibody.

6.6 Alteration of the Expression of Proteins in NK Cells

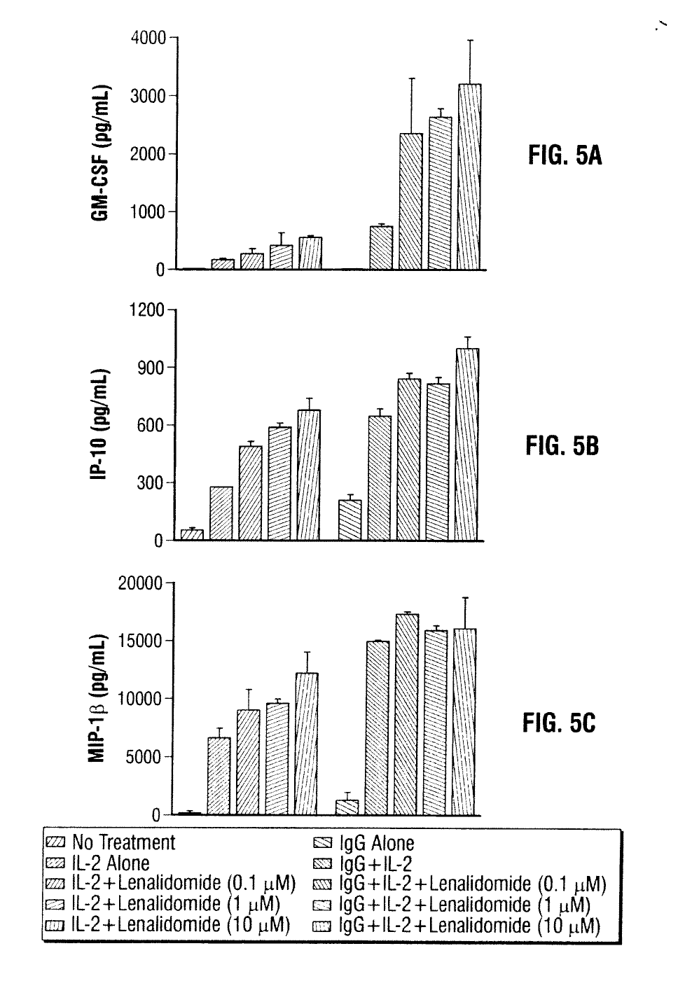

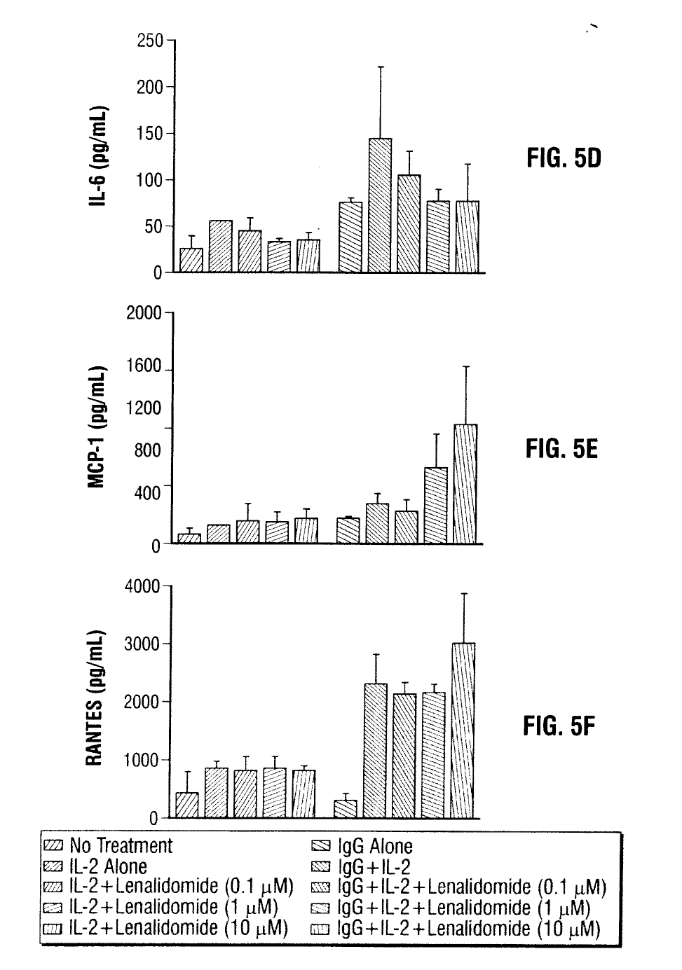

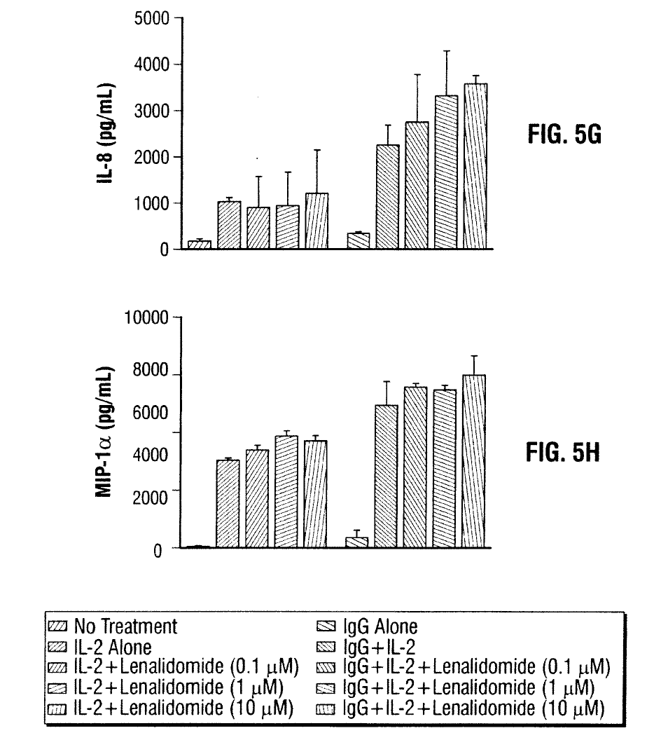

As shown herein, immunomodulatory compounds, either alone or in combination with other agents, can alter the expression of several growth factors. The effect of varying concentrations of the immunomodulatory compound lenalidomide, in combination with IgG and the cytokine IL-2, on the expression of various growth factors and cell cycle proteins is shown in FIG. 5. NK Cells were treated with IL-2, IgG, lenalidomide (0.1 μm, 1 μm, or 10 μm), or combinations of each, and the levels of the proteins GM-CSF, IL-6, IL-8, IP-10, MCP-1, MIP-1α, MIP-1β, and RANTES were subsequently measured.

Lenalidomide increased NK cell expression of IL-8, monocyte chemotactic protein-1 (MCP-1), RANTES, IP-10, and granulocyte-macrophage colony-stimulating factor (GM-CSF), but decreased expression of IL-6 (FIG. 5) In some cases, marked effects were observed in the absence of the antibody.

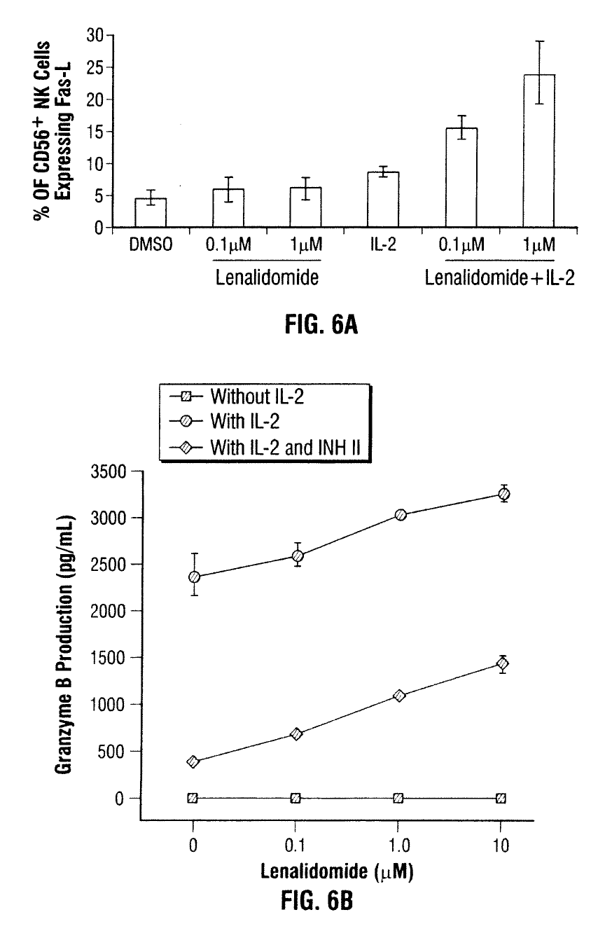

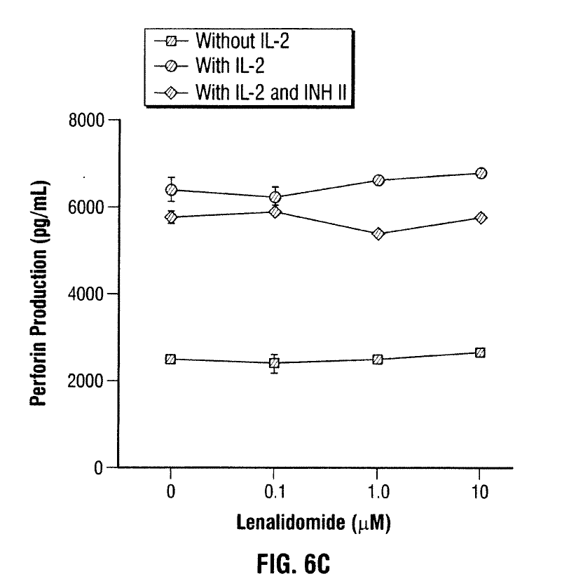

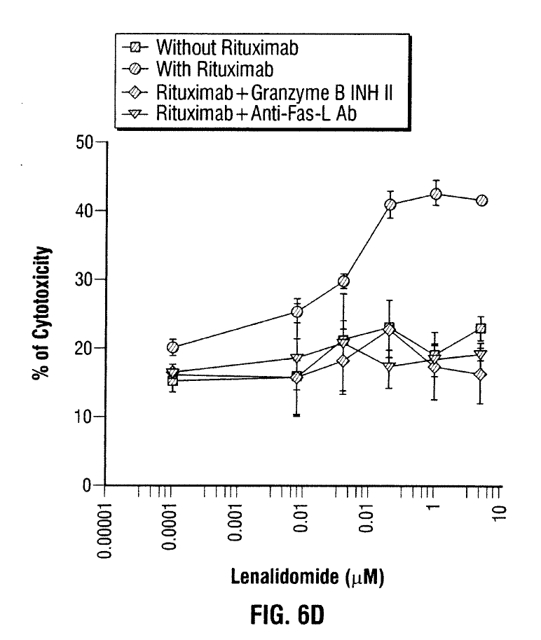

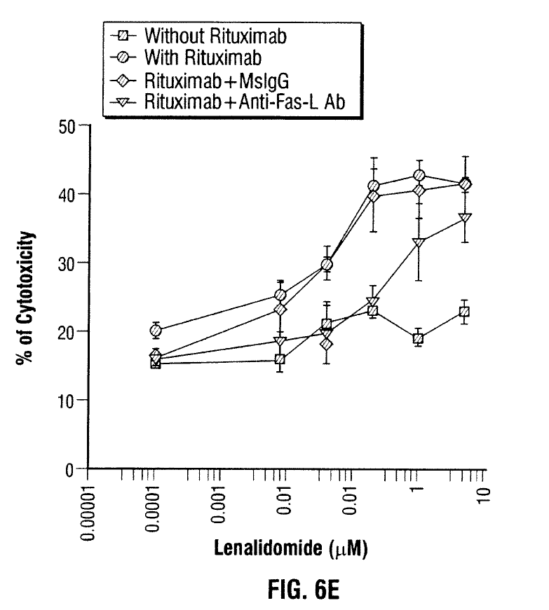

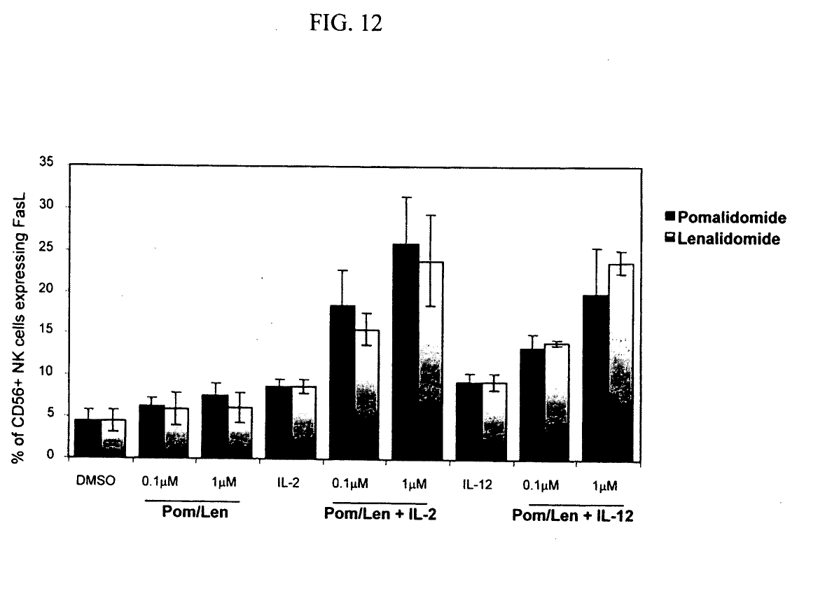

The combination of lenalidomide (10 μM) together with immobilized IgG increased the percentage of CD56+ NK cells expressing Fas-L from <5% to >10% (FIG. 6A). IL-2 (10 ng/mL) treatment with immobilized IgG also increased the NK cell Fas-L expression comparable to lenalidomide treatment. Moreover, the combination of lenalidomide (0.1-10 μM), IL-2 (10 ng/mL), and immobilized IgG enhanced the NK cell Fas-L expression to a level greater than that achieved by either agent alone, demonstrating partially additive responses at 0.1 and 10 μlenalidomide (FIG. 6A). Lenalidomide (1 μM) in conjunction with IL-2 and immobilized IgG demonstrated a synergistic enhancement of NK cell Fas-L expression of >2-fold (from <2% to 25%). The results were similar for the immunomodulatory compound pomalidomide (FIG. 12).

Lenalidomide addition enhanced granzyme B production over that observed with IL-2 alone in a dose-dependent manner. Lenalidomide addition had no effect on perforin production, however (FIGS. 6B and 6C). The granzyme B inhibitor II (40 μM/mL, Calbiochem), added to the NK cells 1 hour prior to lenalidomide and IL-2 treatment, abrogated the effect of IL-2 on granzyme B production. The granzyme B inhibitor II was inactive against the IL-2-stimulated perforin production. ADCC was partially prevented by the addition of anti-Fas-L (FIG. 6D), and was totally prevented in the presence of a granzyme inhibitor (FIG. 6E).

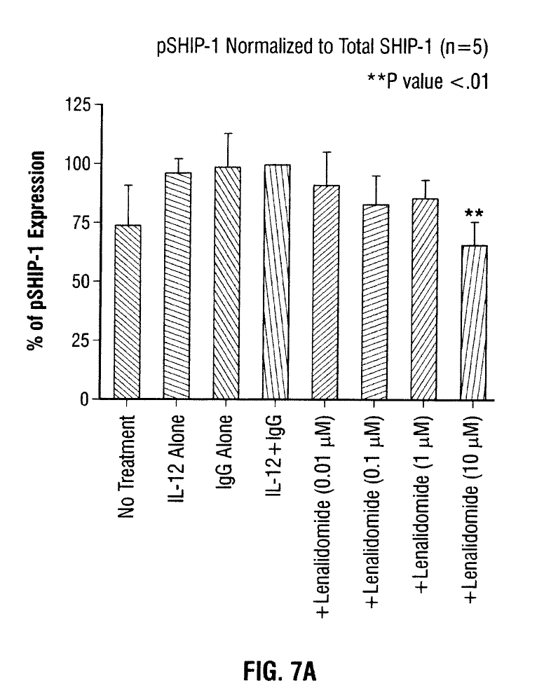

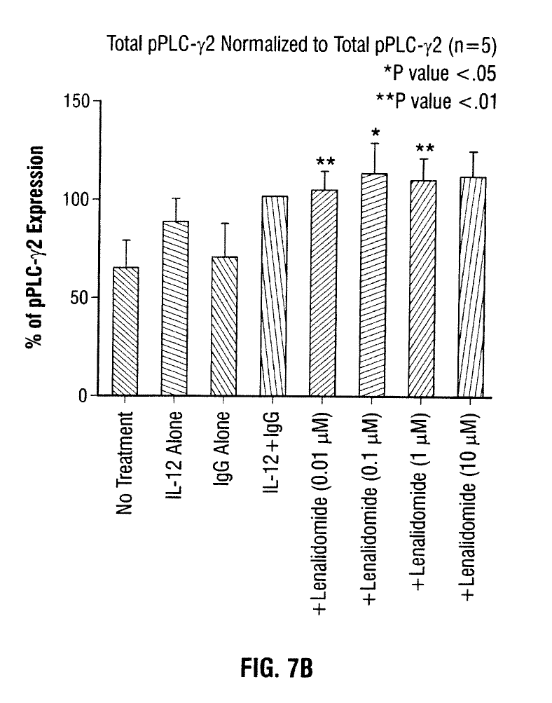

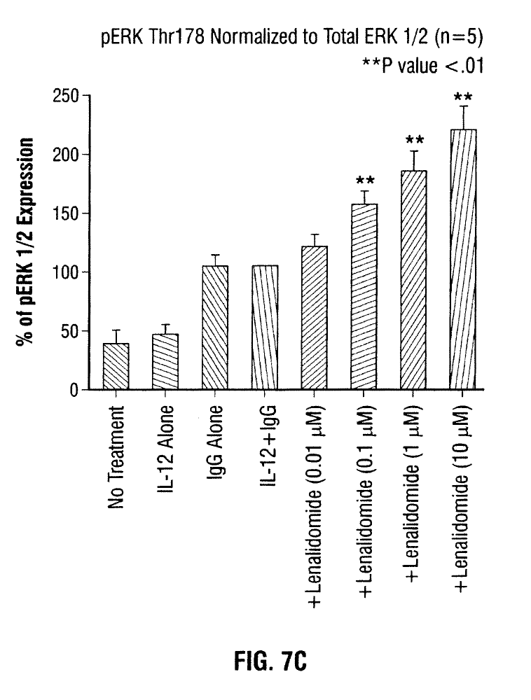

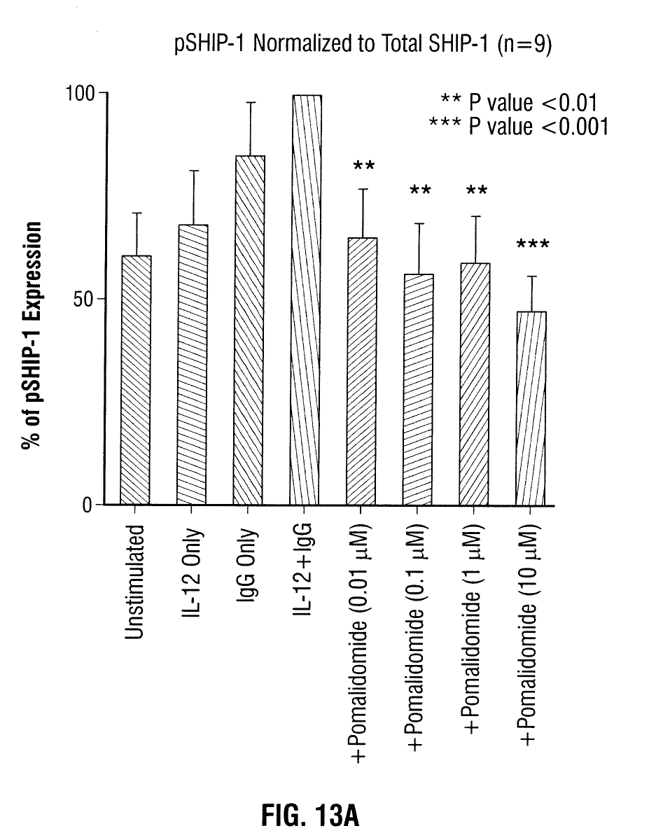

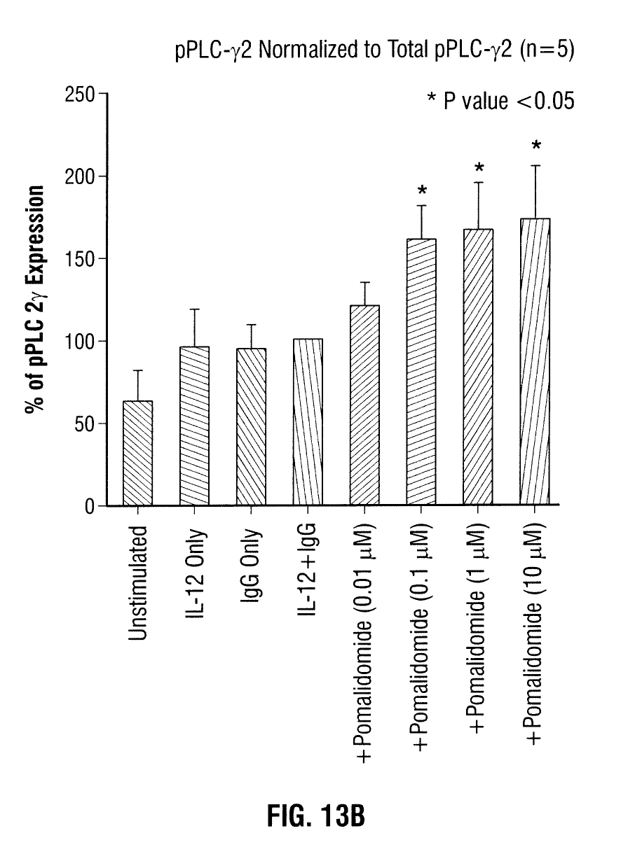

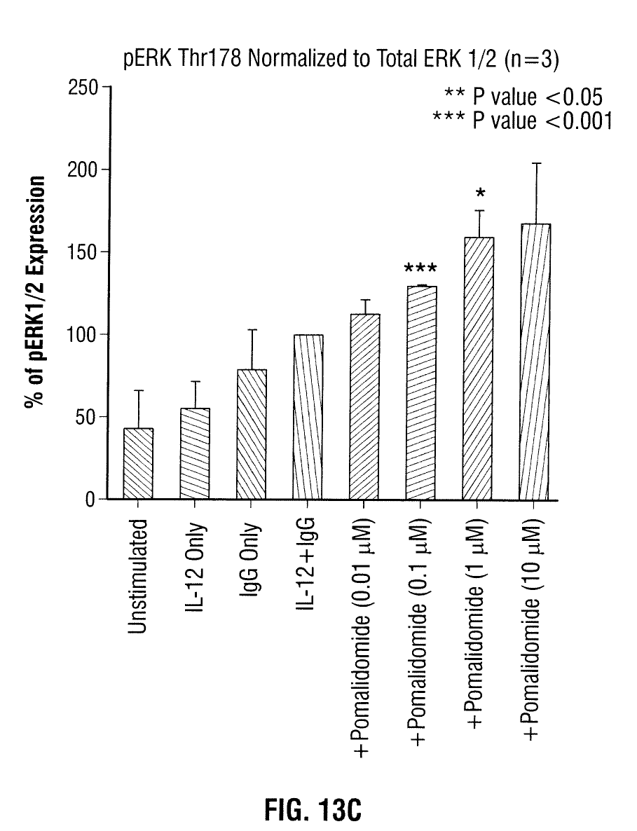

An immunoblot analysis demonstrated that lenalidomide (10 μM) decreased levels of Src homology-2 containing inositol 5′-phosphatase 1 (phospho-SHIP-1) (FIG. 7A). A similar western blot analysis demonstrated that pomalidomide also decreased levels of phospho-SHIP-1 (FIG. 13A). SHIP-1 is a negative regulator of cell proliferation, survival, and activation and also a negative regulator of Fc-γ and cytokine receptor-mediated signaling (Parihar et al., 2005, Cancer Res. 65:9099-9107). The inhibition of phospho-SHIP-1 increases IFN-γ release following NK cell stimulation with IgG and IL-12 (Parihar et al., 2005, Cancer Res. 65:9099-9107, which is incorporated by reference herein in its entirety) and thus may contribute to the mechanism of action of lenalidomide.

Additionally, administration of the immunomodulatory compound lenalidomide was capable of increasing both pPLC-γ2 and pERK levels (FIGS. 7B and 7C). Increase in the levels of these proteins were also observed in connection with pomalidomide (FIGS. 13A and 13B). PLC-γ2 is involved in the maturation and activation of NK cells and triggers the release of lytic granules (Regunathan et al., 2006, Jour. Immunol 175:749-754, which is incorporated by reference herein in its entirety). PLC-γ2 also induces IFN-γ production and is necessary for cytotoxicity and chemokine production.

6.7 Enhancement of NK Cell Cytotoxicity

Enhancement of NK cell cytotoxicity by immunomodulatory compounds lenalidomide and pomalidomide was assessed following procedures substantially similar to those described in Section 6.3, above.

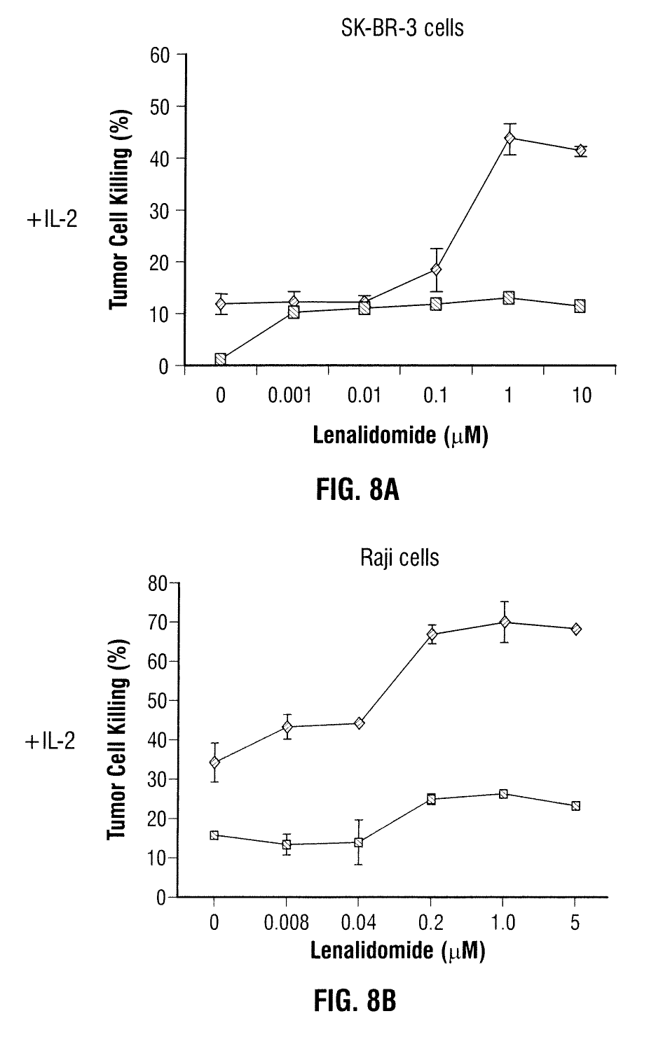

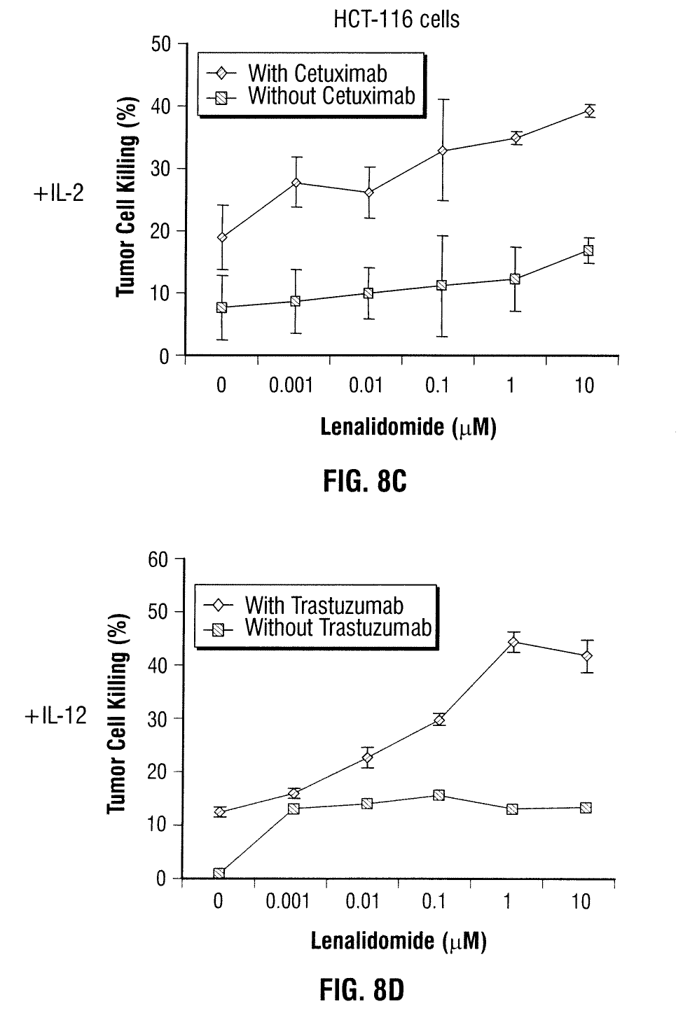

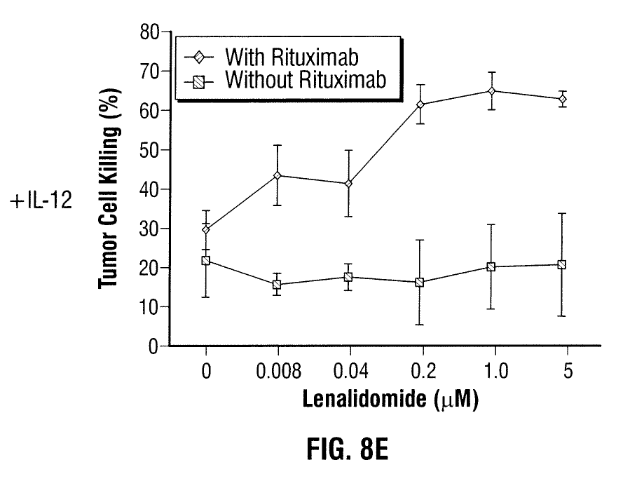

As shown in FIG. 8, the results demonstrated that lenalidomide significantly increases the NK cell cytotoxicity against Trastuzumab-coated SK-BR-3 cells in the presence of IL-2 (FIG. 8A) or IL-12 (FIG. 8D). In addition, it was shown that lenalidomide significantly increases the NK cell cytotoxicity against Rituximab-coated Raji cells in the presence of IL-2 (FIG. 8B) or IL-12 (FIG. 8E), and against Cetuximab-coated HCT-116 cells in the presence of IL-2 (FIG. 8C).

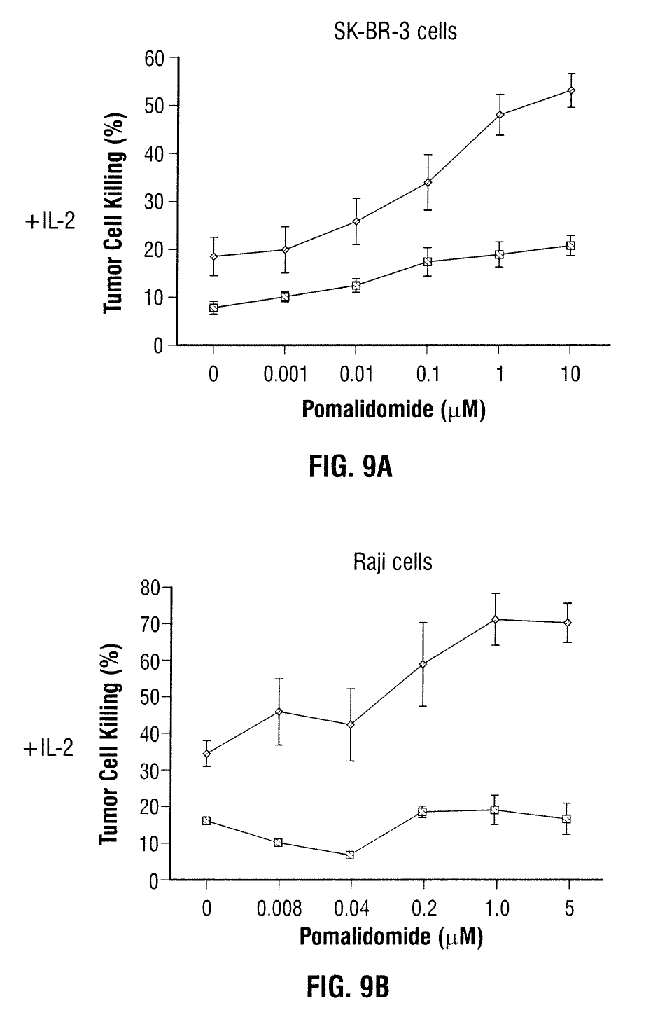

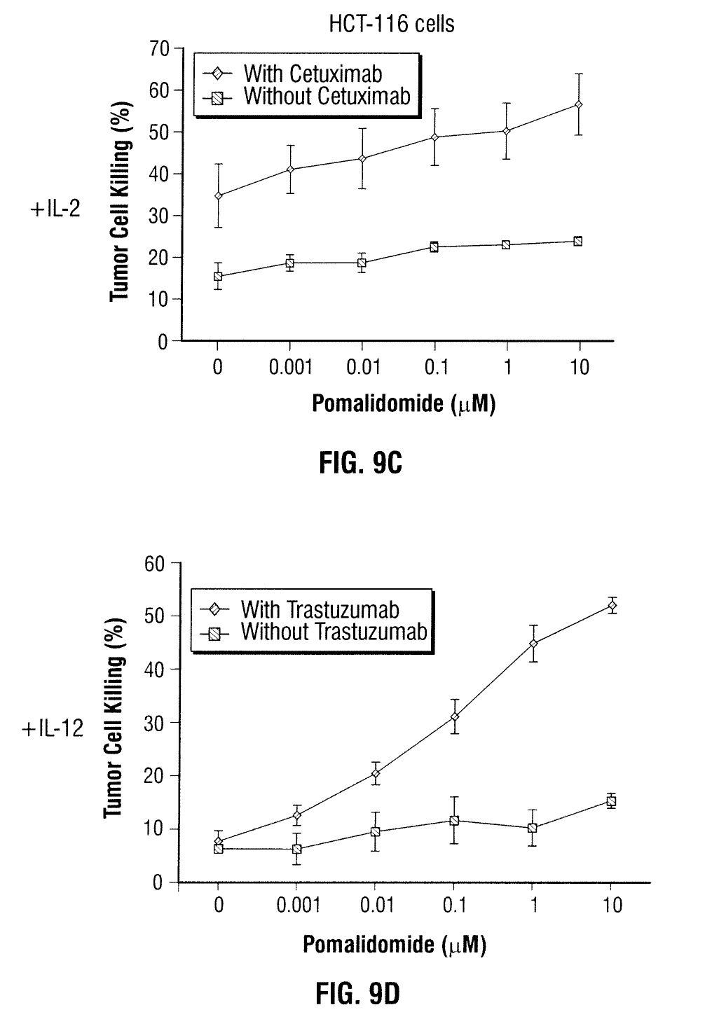

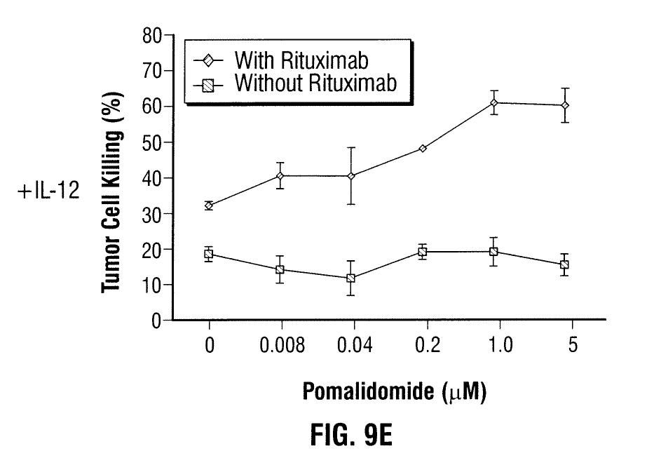

As shown in FIG. 9, similar results were obtained for the immunomodulatory compound pomalidomide. The results demonstrated that pomalidomide significantly increases the NK cell cytotoxicity against Trastuzumab-coated SK-BR-3 cells in the presence of IL-2 (FIG. 9A) or IL-12 (FIG. 9D). In addition, it was shown that pomalidomide significantly increases the NK cell cytotoxicity against Rituximab-coated Raji cells in the presence of IL-2 (FIG. 9B) or IL-12 (FIG. 9E), and against Cetuximab-coated HCT-116 cells in the presence of IL-2 (FIG. 9C).

6.8 Enhancement of Chemokine/Cytokine Production

The effects of immunomodulatory compounds lenalidomide and pomalidomide on NK cell chemokine/cytokine production in response to Trastuzumab-coated SK-BR3 cells were assessed using procedures substantially similar to those described in Section 6.6., above.

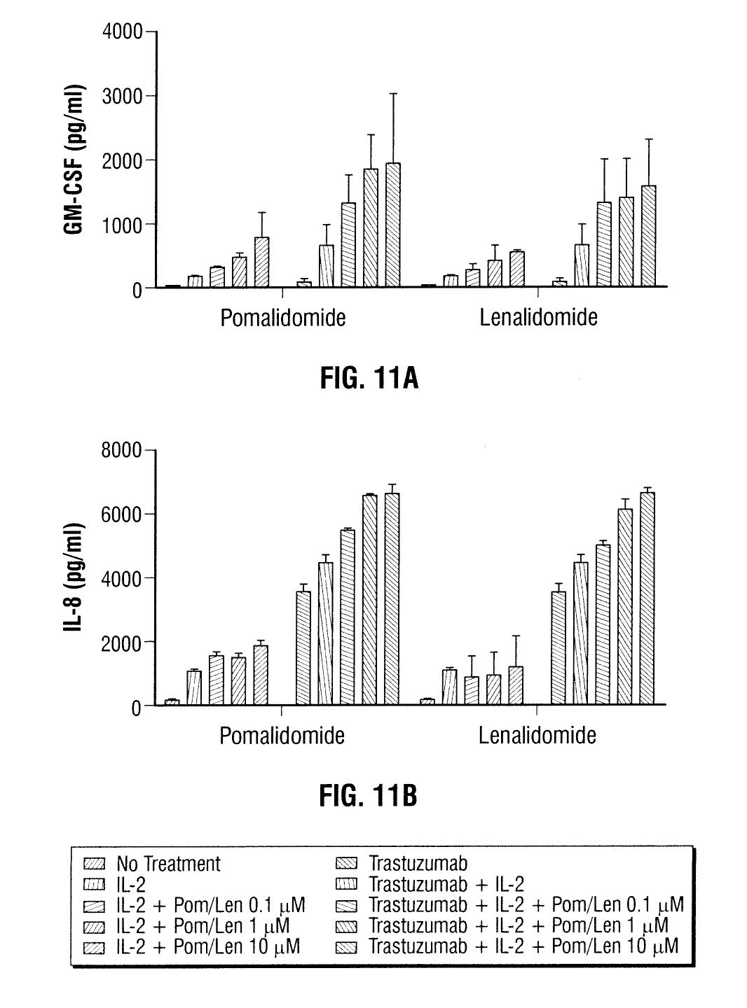

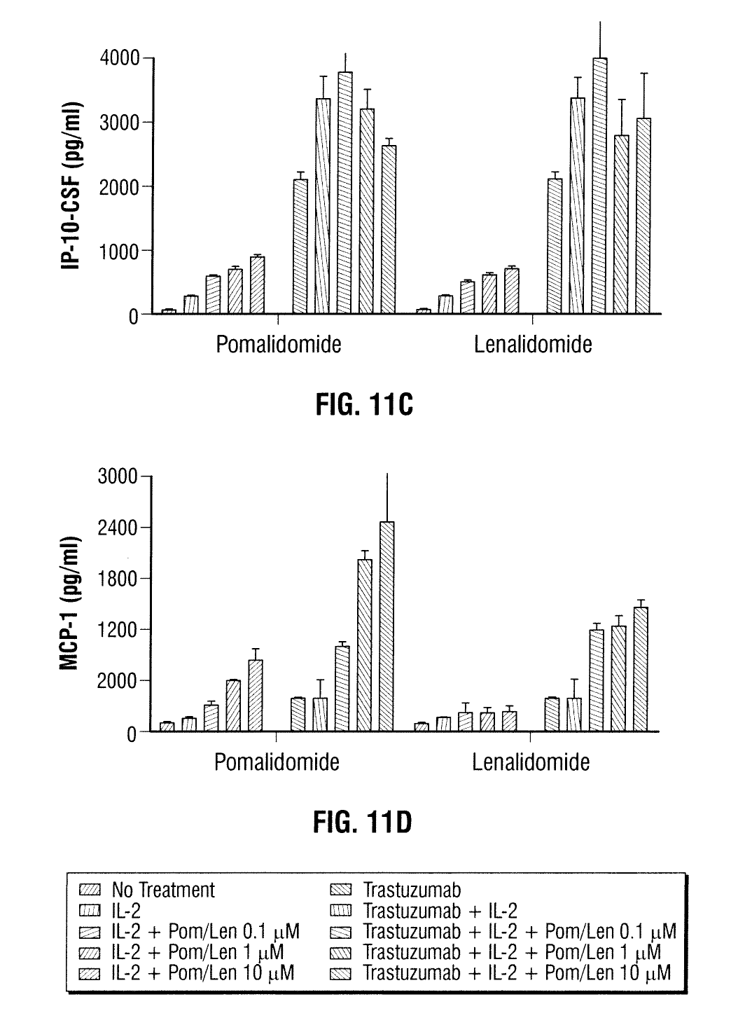

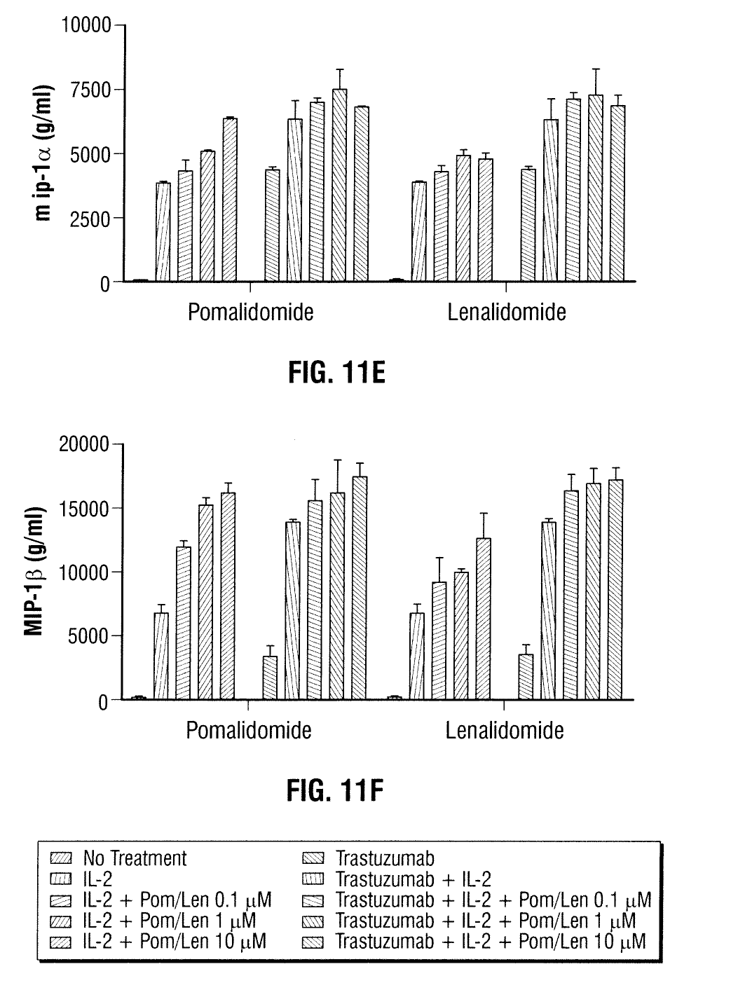

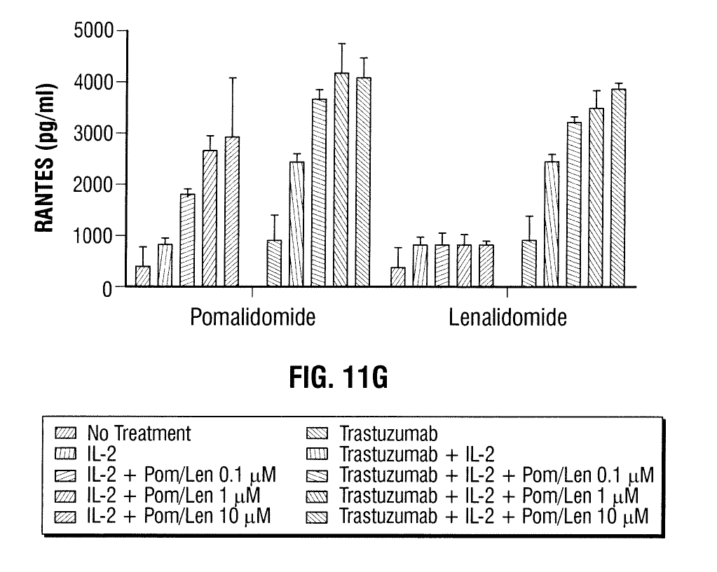

As shown in FIG. 11, both lenalidomide and pomaildomide dose-dependently enhanced the production of GM-CSF (FIG. 11A), IL-8 (FIG. 11B), IP-10 (FIG. 11C), MCP-1 (FIG. 11D), MIP-1α (FIG. 11E), and RANTES (FIG. 11G), but not MIP-1β (FIG. 11F) in the presence of immobilized IgG and IL-2. For most of the chemokines/cytokines, the enhancement in production by the immunomodulatory compound was much more significant when SK-BR3 cells were coated with Trastuzumab. Lenalidomide or pomalidomide alone did not induce the production of any chemokines/cytokines.

All of the references cited herein are incorporated by reference in their entirety. While the description is provided with respect to the particular embodiments, it will be apparent to those skilled in the art that various changes and modifications may be made as recited by the appended claims.

The embodiments provided herein described above are intended to be merely exemplary, and those skilled in the art will recognize, or will be able to ascertain using no more than routine experimentation, numerous equivalents of specific compounds, materials, and procedures. All such equivalents are considered to be within the scope of the disclosure and are encompassed by the appended claims.

What is claimed:

1. A method of treating cancer in a patient, comprising administering an immunomodulatory compound and immunoglobulin G (IgG).

2. The method of claim 1, further comprising administering IL-2 or IL-12.

3. The method of claim 1, wherein the immunomodulatory compound is a compound of formula I, II, III, IV, V, VI, VII, VIII, IX, X, XI, XII, XIII, XIV, XV, XVI, XVII, XVIII or a pharmaceutically acceptable salt, solvate, or stereoisomer thereof: 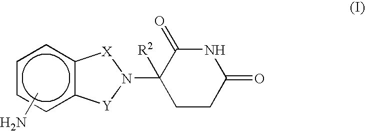
wherein:
one of X and Y is C═O, the other of X and Y is C═O or CH₂;
R² is hydrogen or lower alkyl; 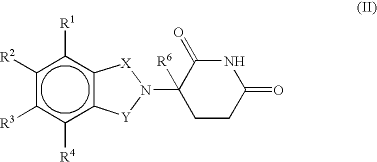
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms, or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzyl, or halo;
provided that R⁶ is other than hydrogen if X and Y are C═O and (i) each of R¹, R², R³, and R⁴ is fluoro or (ii) one of R¹, R², R³, or R⁴ is amino; 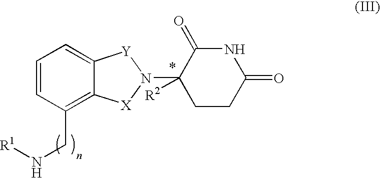
wherein:
one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
* is a chiral-carbon center; 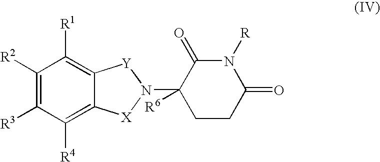
wherein:
one of X and Y is C═O and the other is CH₂ or C═O;
R is H or CH₂OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;
R¹⁰ is hydrogen, alkyl of to 8 carbon atoms, or phenyl; and
* represents a chiral-carbon center; 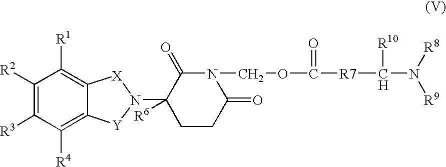
wherein:
one of X and Y is C═O and the other of X and Y is C═O or CH₂;
(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen or alkyl of 1 to 8 carbon atoms;
R⁶ is hydrogen, alkyl of 1 to 8 carbon atoms, benzo, chloro, or fluoro;
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S—, or —NH—; and
R¹⁰ is hydrogen, alkyl of to 8 carbon atoms, or phenyl; 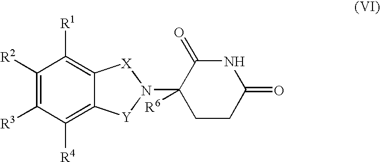
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and
R⁶ is hydrogen, alkyl of 1 to 8 carbon atoms, benzo, chloro, or fluoro; 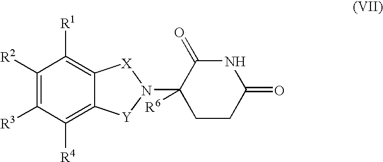
wherein:
one of X and Y is C═O and the other of X and Y is C═O or CH₂;
(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;
R⁵ is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and
R⁶ is alkyl of 1 to 8 carbon atoms, benzo, chloro, or fluoro; 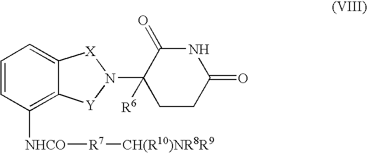
wherein:
one of X and Y is C═O and the other of X and Y is C═O or CH₂;
R⁶ is hydrogen, alkyl of 1 to 8 carbon atoms, benzyl, chloro, or fluoro;
R⁷ is m-phenylene, p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and
R¹⁰ is hydrogen, alkyl of 1 to 8 carbon atoms, or phenyl; 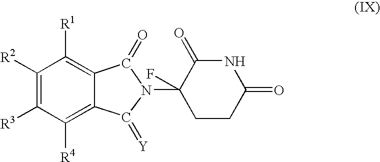
wherein:
Y is oxygen or H² and
each of R¹, R², R³, and R⁴, independently of the others, is hydrogen, halo, alkyl of 1 to 4 carbon atoms, alkoxy of 1 to 4 carbon atoms, or amino; 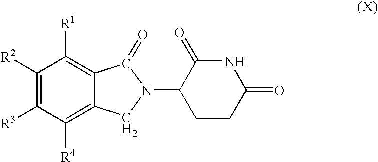
wherein:
each of R¹, R², R³, and R⁴, independently of the others, is halo, alkyl of 1 to 4 carbon atoms, or alkoxy of 1 to 4 carbon atoms; 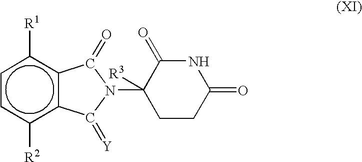
wherein:
Y is oxygen or H₂,
a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and
R³ is hydrogen, alkyl, or benzyl; 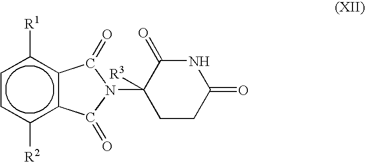
wherein:
a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl;
the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl; and
R³ is hydrogen, alkyl of from 1 to 4 carbon atoms, or benzyl; 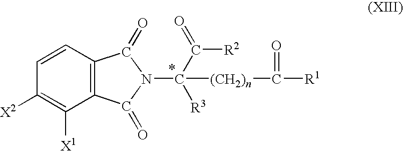
wherein:
when n is not zero and R¹ is not the same as R², C* is a center of chirality;
one of X¹ and X² is amino, nitro, alkyl of one to six carbons, or NH—Z, and the other of X¹ or X² is hydrogen;
each of R¹ and R² independent of the other, is hydroxy or NH—Z; R³ is hydrogen, alkyl of one to six carbons, halo, or haloalkyl;
Z is hydrogen, aryl, alkyl of one to six carbons, formyl, or acyl of one to six carbons; and
n has a value of 0, 1, or 2;
provided that if X¹ is amino, and n is 1 or 2, then R¹ and R² are not both hydroxy; 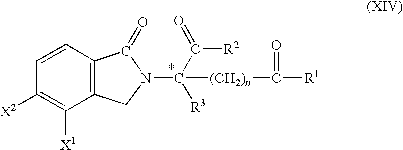
wherein:
when n is not zero and R¹ is not R², C* is a center of chirality;
one of X¹ and X² is amino, nitro, alkyl of one to six carbons, or NH—Z, and the other of X¹ or X² is hydrogen;
each of R¹ and R² independent of the other, is hydroxy or NH—Z; R³ is alkyl of one to six carbons, halo, or hydrogen;
Z is hydrogen, aryl or an alkyl or acyl of one to six carbons; and
n has a value of 0, 1, or 2; 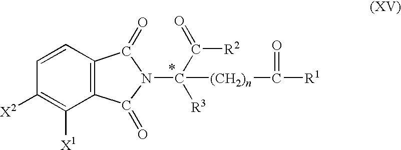
wherein:
when n is not zero and R¹ is not R², C* is a center of chirality;
one of X¹ and X² is amino, nitro, alkyl of one to six carbons, or NH—Z, and the other of X¹ or X² is hydrogen;
each of R¹ and R² independent of the other, is hydroxy or NH—Z; R³ is alkyl of one to six carbons, halo, or hydrogen;
Z is hydrogen, aryl, or an alkyl or acyl of one to six carbons; and
n has a value of 0, 1, or 2; 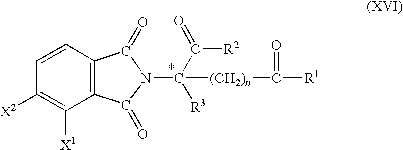
wherein:
one of X¹ and X² is nitro, or NH—Z, and the other of X¹ or X² is hydrogen;
each of R¹ and R², independent of the other, is hydroxy or NH—Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality; 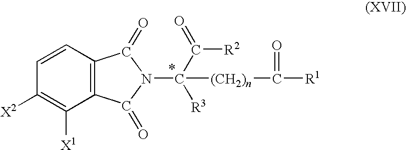
wherein:
one of X¹ and X² is alkyl of one to six carbons;
each of R¹ and R², independent of the other, is hydroxy or NH—Z;
R³ is alkyl of one to six carbons, halo, or hydrogen;
Z is hydrogen, phenyl, an acyl of one to six carbons, or an alkyl of one to six carbons;
n has a value of 0, 1, or 2; and
if —COR² and —(CH₂)ₙCOR¹ are different, C* is a center of chirality; 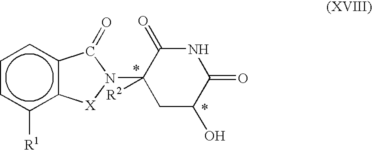
wherein:
the * carbons are centers of chirality;
X is —C(O)— or —CH₂—;
R¹ is alkyl of 1 to 8 carbon atoms or —NHR³;
R² is hydrogen, alkyl of 1 to 8 carbon atoms, or halogen; and
R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴, wherein
R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

4. The method of claim 1, wherein said immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

5. The method of claim 1, wherein said immunomodulatory compound is administered after administration of said IgG.

6. The method of claim 5, wherein the immunomodulatory compound is administered from about 30 minutes to about 2 weeks after the administration of said IgG.

7. The method of claim 6, wherein the immunomodulatory compound is administered from about 1 hour to about 2 days after the administration of said IgG.

8. The method of claim 1, wherein said IgG is human serum IgG.

9. The method of claim 8, wherein said IgG is purified human serum IgG.

10. The method of claim 1, wherein said IgG is a monoclonal or polyclonal antibody.

11. The method of claim 1, wherein said IgG is a chimeric antibody.

12. The method of claim 1, wherein said cancer is NHL or CLL.

13. A method of treating cancer in a subject, comprising administering an immunomodulatory compound to said subject prior to administration of Rituximab to said subject.

14. The method of claim 13, wherein the immunomodulatory compound is administered from about 30 minutes to about 2 weeks prior to the administration of said Rituximab.

15. The method of claim 14, wherein the immunomodulatory compound is administered from about 1 hour to about 2 days prior to the administration of said Rituximab.

16. The method of claim 13, wherein said immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

17. The method of claim 13, further comprising administering IL-2 or IL-12 to said subject.

18. The method of claim 13, further comprising administering human serum IgG to said subject.

19. The method of claim 13, wherein said cancer is lymphoma.

20. A method of treating cancer in a subject, comprising administering an immunomodulatory compound to said subject prior to administration of Trastuzumab to said subject.

21. The method of claim 20, wherein the immunomodulatory compound is administered from about 30 minutes to about 2 weeks prior to the administration of said Trastuzumab.

22. The method of claim 21, wherein the immunomodulatory compound is administered from about 1 hour to about 2 days prior to the administration of said Trastuzumab.

23. The method of claim 20, wherein said immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

24. The method of claim 20, further comprising administering IL-2 or IL-12 to said subject.

25. The method of claim 20, further comprising administering human serum IgG to said subject.

26. The method of claim 20, wherein said cancer is breast cancer.

27. A method of treating cancer in a subject, comprising administering an immunomodulatory compound to said subject prior to administration of Cetuximab to said subject.

28. The method of claim 27, wherein the immunomodulatory compound is administered from about 30 minutes to about 2 weeks prior to the administration of said Cetuximab.

29. The method of claim 28, wherein the immunomodulatory compound is administered from about 1 hour to about 2 days prior to the administration of said Cetuximab.

30. The method of claim 27, wherein said immunomodulatory compound is 1-oxo-2-(2,6-dioxopiperidin-3-yl)-4-aminoisoindoline or 1,3-dioxo-2-(2,6-dioxopiperidin-3-yl)-4-aminoisoindoline.

31. The method of claim 27, further comprising administering IL-2 or IL-12 to said subject.

32. The method of claim 27, further comprising administering human serum IgG to said subject.

33. The method of claim 27, wherein said cancer is colorectal cancer.
